(19) United States
(12) Patent Application Publication
(10) Pub. No.: US 2025/0382280 A1
(43) Pub. Date: Dec. 18, 2025

(54) PYRAZOLE DERIVATIVES FOR THE INHIBITION OF PHAGOCYTOSIS

(71) Applicants: Canadian Blood Services, Ottawa (CA); University Health Network, Toronto (CA)

(72) Inventors: Donald R. Branch, Toronto (CA); Lakshmi P. Kotra, Toronto (CA); Melissa Lewis-Bakker, Toronto (CA)

(21) Appl. No.: 18/876,279

(22) PCT Filed: Jun. 28, 2023
(86) PCT No.: PCT/CA2023/050888

§ 371 Date: Dec. 18, 2024

Related U.S. Application Data
(60) Provisional application No. 63/356,170, filed on Jun. 28, 2022.

(51) Int. Cl. C07D 403/12; A61K 31/381; A61K 31/4155; A61K 31/426; C07D 407/04; C07D 409/14; C07D 417/14; C07D 487/04; C07D 495/04

(52) U.S. Cl. CPC C07D 403/12; A61K 31/381; A61K 31/4155; A61K 31/426; C07D 407/04; C07D 409/14; C07D 417/14; C07D 487/04; C07D 495/04

(57) ABSTRACT

There is provided a method of treating an immune cytopenia in a subject in need thereof, the method comprising administering a therapeutically effective amount of a compound of formula (I) wherein R₁, R₂, R₃ and R₄ are as defined herein.

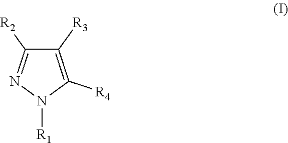

### CROSS REFERENCE TO A RELATED APPLICATION

This disclosure claims the priority of U.S. provisional application No. 63/356,170 filed on Jun. 28, 2022, which is incorporated herein by reference in its entirety.

### TECHNICAL FIELD

This disclosure relates to the field of small molecule inhibitors, such as pyrazole derivatives, which are useful, for example, for the inhibition of phagocytosis in a subject having a condition, such as an immune cytopenia, where the inhibition of phagocytosis can treat or alleviate the symptoms of that condition.

### BACKGROUND OF THE ART

Immune cytopenias are conditions in which people generate antibodies against certain types of hematopoietic cells in their blood. Cells get coated with antibodies under these circumstances and are then identified by the Fcγ receptors (FcγR) on the membrane of mononuclear phagocytes. Such recognition by monocyte-macrophages results in extravascular hemolysis in the spleen and/or liver macrophages due to FcγR-mediated phagocytosis. Affected individuals can face severe and sometimes even life-threatening complications due to this process. Immune cytopenias have many categories, including (a) immune thrombocytopenia (ITP; autoimmune disease characterized by increased platelet destruction in the spleen and liver and/or decreased platelet production in the bone marrow); (b) hemolytic disease of the fetus and newborn (HDFN; maternal hemolytic antibodies crossing the placenta); (c) autoimmune hemolytic anemia (AIHA; phagocytosis of autoantibody-coated red blood cells); (d) alloimmune hemolytic anemias such as hemolytic transfusion reaction (HTR; phagocytosis of donor red blood cells due to preformed hemolytic alloantibodies to the donor red blood cell antigens); (e) delayed hemolytic transfusion reaction (DHTR; development of hemolytic alloantibodies following transfusion); and (f) autoimmune neutropenia (AlN) associated with autoantibodies produced against neutrophils, mainly affecting children.

What all immune cytopenias have in common is the destruction of the particular blood cells opsonized with antibody by FcγR-mediated phagocytosis. Thus, the development of small molecule agents (drugs) that would provide blockade of the phagocytosis would ameliorate the various immune cytopenias and, elucidation of such drugs would provide a useful clinical intervention.

Treatment of immune cytopenias with the exception of ITP primarily involves corticosteroids (dexamethasone, prednisone) and monoclonal anti-CD20 (rituximab). For ITP, there are a few additional therapeutics that are in use as secondary or tertiary therapies. These include splenectomy, thrombopoietin receptor agonists (TPO-RAs; Eltrombopag and Avatrombopag) to stimulate increased platelet production, IVIg and anti-D (mechanism unclear), and spleen tyrosine kinase (Syk) inhibitors (fostamatinib). Most novel therapeutics have targeted ITP and not other immune cytopenias such as AlN, AIHA, HTR, DHTR, or HDFN. There are a number of experimental therapeutics in various stages of development and clinical trials, such as recombinant Fc multimers and inhibitors of the neonatal Fc receptor (FcRn) that may have efficacy in immune cytopenias other than ITP. Accordingly, a treatment that can be applied to all immune cytopenias or conditions where phagocytosis is part of the pathophysiology is desired.

### SUMMARY

There is provided a compound of formula I, defined as follows:

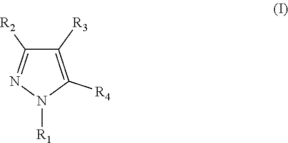

R₁ is phenyl or ethyl;
R₂ is selected from the group consisting of H, —C(O)—O—CH₂—CH₃ and

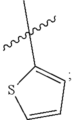 [SMILES: *c1cccs1]

R₃ is H,

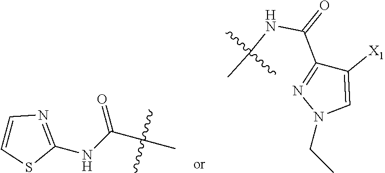

wherein X₁ is H, methyl or a halogen such as Br, F or Cl; and
R₄ is —CN,

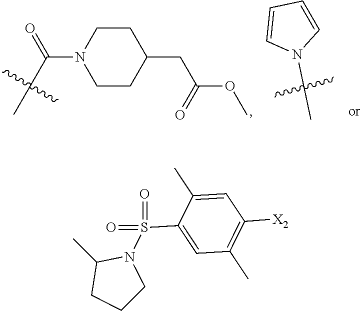

wherein X₂ is H or methyl;
when R₁ is phenyl
R₂ is —C(O)—O—CH₂—CH₃, R₃ is

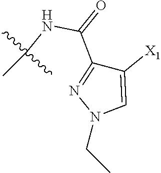

wherein X₁ is H, methyl or a halogen such as Br, F or Cl, and R₄ is —CN;
R₂ is

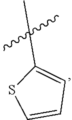 [SMILES: *c1cccs1]

R₃ is H and R₄ is

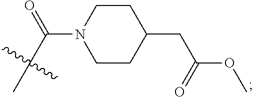 [SMILES: *C(=O)N1CCC(CC(=O)OC)CC1]

or
R₂ is H, R₃ is

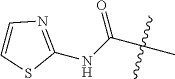 [SMILES: *C(=O)Nc1nccs1]

### and R₄ is

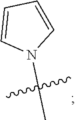 [SMILES: *n1cccc1]

and when R₁ is ethyl R₂ and R₃ are H and R₄ is

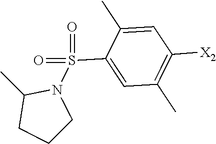

wherein X₂ is H or methyl.

In some embodiments, the compound of formula I is

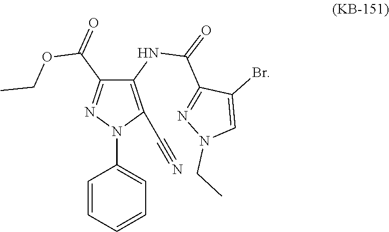 [SMILES: CCOC(=O)c1nn(-c2ccccc2)c(C#N)c1NC(=O)c1nn(CC)cc1Br]

In some embodiments, the compound of formula I is

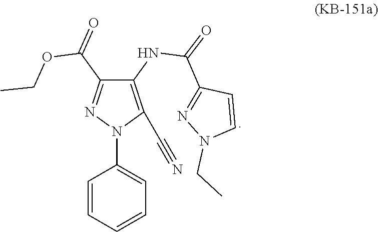 [SMILES: CCOC(=O)c1nn(-c2ccccc2)c(C#N)c1NC(=O)c1ccn(CC)n1]

In some embodiments, the compound of formula I is

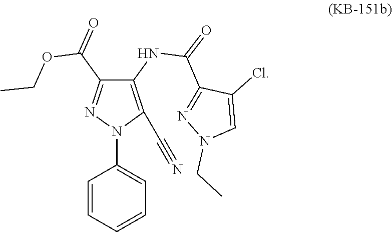 [SMILES: CCOC(=O)c1nn(-c2ccccc2)c(C#N)c1NC(=O)c1nn(CC)cc1Cl]

In some embodiments, the compound of formula I is

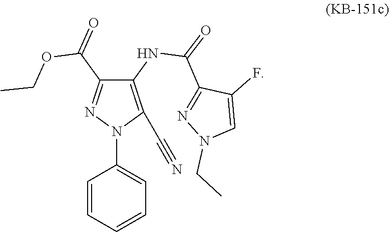 [SMILES: CCOC(=O)c1nn(-c2ccccc2)c(C#N)c1NC(=O)c1nn(CC)cc1F]

In some embodiments, the compound of formula I is

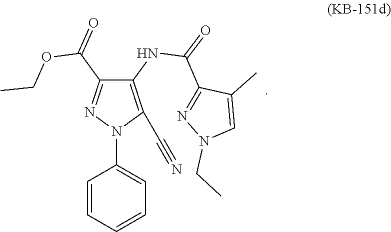 [SMILES: CCOC(=O)c1nn(-c2ccccc2)c(C#N)c1NC(=O)c1nn(CC)cc1C]

In some embodiments, the compound of formula I is

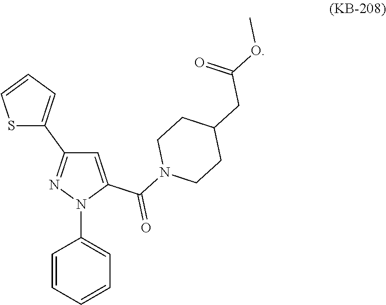 [SMILES: COC(=O)CC1CCN(C(=O)c2cc(-c3cccs3)nn2-c2ccccc2)CC1]

In some embodiments, the compound of formula I is

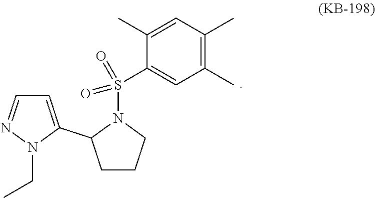 [SMILES: CCn1nccc1C1CCCN1S(=O)(=O)c1cc(C)c(C)cc1C]

In some embodiments, the compound of formula I is

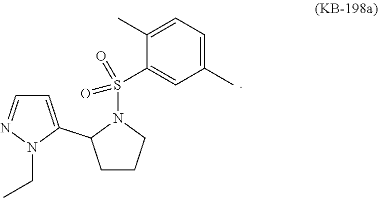 [SMILES: CCn1nccc1C1CCCN1S(=O)(=O)c1cc(C)ccc1C]

In some embodiments, the compound of formula I is

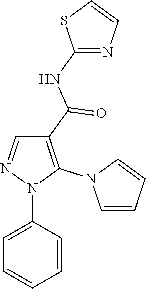 [SMILES: O=C(Nc1nccs1)c1cnn(-c2ccccc2)c1-n1cccc1]

### (KB-210).

In one aspect there is provided a method of treating an immune cytopenia in a subject in need thereof, the method comprising administering a therapeutically effective amount of a compound of formula I. In one aspect there is provided a method of treating an immune cytopenia in a subject in need thereof, the method comprising administering a therapeutically effective amount of a pharmaceutical composition comprising the compound of formula I and a pharmaceutically acceptable excipient.

In one aspect there is provided the use of a compound of formula I for the treatment of an immune cytopenia. In a further aspect, there is provided the use of a pharmaceutical composition comprising the compound of formula I and a pharmaceutically acceptable excipient for the treatment of an immune cytopenia.

In one aspect, there is provided a compound of formula I for the treatment of an immune cytopenia. In a further aspect, there is provided a pharmaceutical composition comprising a pharmaceutically acceptable excipient and a compound of formula I for use in the treatment of an immune cytopenia.

In some embodiments, the immune cytopenia is immune thrombocytopenia, hemolytic disease of the fetus and newborn, autoimmune hemolytic anemia, alloimmune hemolytic anemias, delayed hemolytic transfusion reaction or autoimmune neutropenia.

In a further aspect, there is provided method of dephosphorylating HSP27 in a subject in need thereof, the method comprising administering to the subject in need thereof a therapeutically effective amount of compound KB-151, Kb-151a or KB-208. The cardiovascular disease or the cancer have deregulated HSP27 and where HSP27 is phosphorylated. In some embodiments, the cardiovascular disease is atherosclerosis. In some embodiments, the cancer is prostate cancer, colorectal cancer or breast cancer.

Many further features and combinations thereof concerning the present improvements will appear to those skilled in the art following a reading of the instant disclosure.

### DESCRIPTION OF THE DRAWINGS

FIG. 1 is a graph showing the results of the lactate dehydrogenase (LDH) assay. The Y-axis shows the percentage of cell death. Thimerosal at 100 μM (****P≤0.0001) shows a significant difference compared to untreated control.

FIG. 2 is a graph showing the results of the MTT assay. The Y-axis shows the percentage of cell viability which relates to the cell metabolic activity. Thimerosal at 100 μM (****P≤0.0001), KB-182 (**P≤0.005), and KB-178 (*P≤0.05) show significant differences compared to untreated control.

FIG. 3A is a graph of the inhibition of phagocytosis in function of the concentration of the compound for compound showing the IC₅₀.

FIG. 3B is graph of the inhibition of phagocytosis in function of the concentration of the compound for compound KB-208 showing the IC₅₀.

FIG. 3C is graph of the inhibition of phagocytosis in function of the concentration of the compound for compound KB-198 showing the IC₅₀.

FIG. 3D is graph of the inhibition of phagocytosis in function of the concentration of the compound for compound KB-210 showing the IC₅₀.

FIG. 3E is a graph of the inhibition in function of the concentration of IVIg (dose-inhibitory response of IVIg alone (..-..-) or with addition of KB-208 (-) or KB-151 (----) at their IC₅₀ concentration, 4 μM and 3 μM, respectively, administered along with IVIg at each IVIg concentration used in its titration (arrows)).

FIG. 3F is a graph of the inhibition in function of the concentration of the compound for compounds KB-151, KB-198, KB-208 and KB-210 (respectively •, ▴, ▪, and ♦).

FIG. 3G is a graph of the inhibition of phagocytosis in function of the concentration of the compound for compound KB-151a showing the IC₅₀.

FIG. 3H is a graph of the inhibition of phagocytosis in function of the concentration of the compound for compound KB-151b showing the IC₅₀.

FIG. 3I is a graph of the inhibition of phagocytosis in function of the concentration of the compound for compound KB-151c showing the IC₅₀.

FIG. 3J is a graph of the inhibition of phagocytosis in function of the concentration of the compound for compound KB-151d showing the IC₅₀.

FIG. 4A is a bar graph of the MTT assay results for PBMC, HEPG2, and HEK-293 using a concentration (up to 250 μM) of KB-151. Y-axis shows the percentage of metabolic activity which related to the cell viability. X-axis shows different concentrations of KB-151, Thimerosal at 100 μM as control toxicity (****P≤0.0001), and untreated control.

FIG. 4B is a bar graph of the LDH assay results for PBMC, HEPG2, and HEK-293 using a concentration (up to 250 μM) of KB-151. Y-axis shows the percentage of relative specific death which related to the cell toxicity. X-axis shows different concentrations of KB-151, Thimerosal at 100 μM as control toxicity (****P≤0.0001), and untreated control.

FIG. 4C is a bar graph of the MTT assay results for PBMC, HEPG2, and HEK-293 using a concentration (up to 250 μM) of KB-208. Y-axis shows the percentage of metabolic activity which related to the cell viability. X-axis shows different concentrations of KB-208, Thimerosal at 100 μM as control toxicity (****P≤0.0001), and untreated control.

FIG. 4D is a bar graph of the LDH assay results for PBMC, HEPG2, and HEK-293 using a concentration (up to 250 μM) of KB-208 (b). Y-axis shows the percentage of relative specific death which related to the cell toxicity. X-axis shows different concentrations of KB-208, Thimerosal at 100 μM as control toxicity (****P≤0.0001), and untreated control.

FIG. 5A shows flow cytometry graphs for an apoptosis assay for PBMC cells. The cells were treated with KB-208 and KB-151 at 250 μM concentration for 1 hour. Samples were stained by Annexin V and PI and ran through a SP6800 Spectral Cytometer. (graphs from left to right: untreated, Thimerosal, KB-151 and KB-208).

FIG. 5B shows flow cytometry graphs for an apoptosis assay for HEPG2 cells. The cells were treated with KB-208 and KB-151 at 250 μM concentration for 1 hour. Samples were stained by Annexin V and PI and ran through a SP6800 Spectral Cytometer. (graphs from left to right: untreated, Thimerosal, KB-151 and KB-208).

FIG. 5C shows flow cytometry graphs for an apoptosis assay for HEK-293 cells. The cells were treated with KB-208 and KB-151 at 250 μM concentration for 1 hour. Samples were stained by Annexin V and PI and ran through a SP6800 Spectral Cytometer. (graphs from left to right: untreated, Thimerosal, KB-151 and KB-208).

FIG. 6A is a graph showing the platelet count in function of time in a in vivo dose escalation assay of anti-CD41 antibody immune cytopenia (ITP) mouse model. Balb/c animals were either treated with a daily escalating dose of the anti-CD41 antibody ( [symbol] ) only, or treated with anti-CD41 antibody and 1% DMSO ( [symbol] ), or treated with anti-CD41 antibody and KB-151 ( [symbol] ) at 100 μM twice a day given s.c. at two different sites, on day 2 and day 3 (n=3 for each group).

FIG. 6B is a graph showing the platelet count in function of time in a in vivo dose escalation assay of anti-CD41 antibody immune cytopenia (ITP) mouse model. Balb/c animals were either treated with a daily escalating dose of the anti-CD41 antibody ( [symbol] ) only, or treated with anti-CD41 antibody and 1% DMSO ( [symbol] ) twice a day given s.c. at two different sites, on day 2 and day 3, or treated with anti-CD41 antibody, KB-151 at 100 μM twice a day given s.c. at two different sites, on day 2 and day 3, as well as administer IVIg at 0.5 g/kg on day 2 only through intraperitoneal (I.P) ( [symbol] ), or treated with anti-CD41 antibody and IVIg at 0.5 g/kg on day 2 through I.P ( [symbol] ) (n=3 for each group).

FIG. 7 is a graph showing the effect of different routes of administration of KB-151 in an ITP mice model (C57BL/6) presenting the platelet concentration in function of time for intravenous (IV), subcutaneous (SC). oral or intraperitoneal (IP) injections as well as control conditions.

FIG. 8A is a graph showing the platelet concentration in function of time in an ITP mice model (balb/c) treated with an IP injection of KB-208.

FIG. 8B is a graph showing the platelet concentration in function of time in an ITP mice model (balb/c) treated with an IP injection of KB-151.

FIG. 9 is a graph showing the platelet concentration in function of time in an ITP mice model (CD1) treated with an IP injection of KB-151, KB-208 or both.

FIG. 10A is a bar graph showing the biochemistry of a blood test performed on male mice (balb/c) having received a dosage of 1 mg/kg to intraperitoneally twice daily and two times per week for a duration of 60 days (ALT=alanine transaminase, AST=aspartase transferase, ALP=alkaline phosphatase, TBIL=total bilirubin, TP=total protein, ALB=albumin, CHOL=cholesterol, HDL=high density lipoprotein, LDL=low density lipoprotein, TRIG=triglycerides, PHOS=phosphate, CAL=calcium, GLU=glucose, CRE=creatinine, BUN=blood urea nitrogen). For each analyzed blood component from left to write the bars are wild type mice, mice treated after 30 days and mice treated after 60 days.

FIG. 10B is a bar graph showing the biochemistry of a blood test performed on female mice (balb/c) having received a dosage of 1 mg/kg to intraperitoneally twice daily and two times per week for a duration of 60 days (ALT=alanine transaminase, AST=aspartase transferase, ALP=alkaline phosphatase, TBIL=total bilirubin, TP=total protein, ALB=albumin, CHOL=cholesterol, HDL=high density lipoprotein, LDL=low density lipoprotein, TRIG=triglycerides, PHOS=phosphate, CAL=calcium, GLU=glucose, CRE=creatinine, BUN=blood urea nitrogen). For each analyzed blood component from left to write the bars are wild type mice, mice treated after 30 days and mice treated after 60 days.

FIG. 11A is a bright field microscopy image of anti-D opsonized red blood cells (RBC) cultured in the presence of monocytes in vitro at room temperature.

FIG. 11B is a bright field microscopy image of anti-D opsonized red blood cells (RBC) cultured in the presence of monocytes treated with IVIg (6.5 μM) in vitro at room temperature.

FIG. 11C is a bright field microscopy image of anti-D opsonized red blood cells (RBC) cultured in the presence of monocytes treated with KB-151 (100 μM) in vitro at room temperature.

FIG. 11D is a bright field microscopy image of anti-D opsonized red blood cells (RBC) cultured in the presence of monocytes treated with KB-151 (100 μM) in vitro at room temperature.

FIG. 11E is a fluorescence microscopy image of FIG. 11A showing the binding of monocytes with fluorescently labeled anti-D opsonized RBCs.

FIG. 11F is a fluorescence microscopy image of FIG. 11B showing the absence of binding of monocytes with fluorescently labeled anti-D opsonized RBCs thereby indicating the absence of phagocytosis.

FIG. 11G is a fluorescence microscopy image of FIG. 11C showing the absence of binding of monocytes with fluorescently labeled anti-D opsonized RBCs thereby indicating the absence of phagocytosis.

FIG. 11H is a fluorescence microscopy image of FIG. 11D showing the absence of binding of monocytes with fluorescently labeled anti-D opsonized RBCs thereby indicating the absence of phagocytosis.

FIG. 12 is a bar graph of the mean fluorescence intensity (MFI) showing the expression of FcγR 1, II and III on monocytes untreated or treated with KB-151 or KB-208.

FIG. 13A is a bar graph showing the effect of KB-151 and KB-208 on the level of ph-HSP27 (i.e. phosphorylate HSP27) in the absence of red blood cells, in the presence of un opsonized red blood cells (un-ops) and in the presence of opsonized red blood cells (ops).

FIG. 13B is a western blot of the conditions of FIG. 13A, i.e. the detection of ph-HSP7.

FIG. 14 is a graph showing that KB-208 and KB-151 activate PP2A (increase its phosphatase activity) which is the serine phosphatase that dephosphorylates HSP27.

### DETAILED DESCRIPTION

There is provided a compound of formula I for inhibiting phagocytosis.

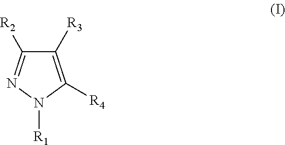

R₁ is phenyl or ethyl, R₂ is selected from the group consisting of H, —C(O)—O—CH₂—CH₃ and

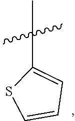 [SMILES: *c1cccs1]

### R₃ is H,

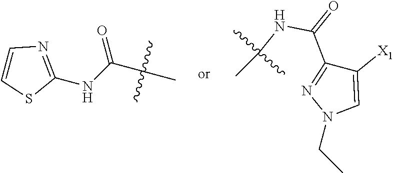

wherein X₁ is H, methyl or a halogen such as Br, F or Cl, and R₄ is —CN,

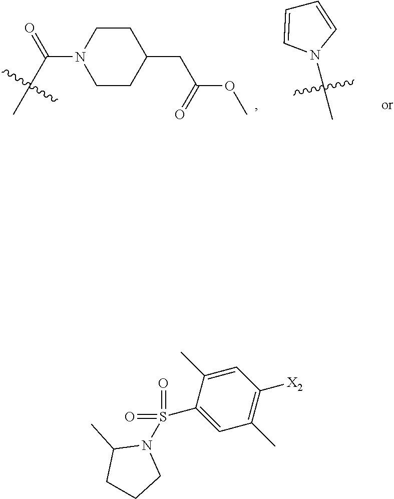

wherein X₂ is H or methyl. When R₁ is phenyl then R₂ is —C(O)—O—CH₂—CH₃, R₃ is

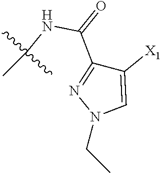

wherein X₁ is H, methyl or a halogen such as Br, F or Cl, and R₄ is —CN; or R₂ is

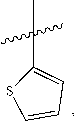 [SMILES: *c1cccs1]

R₃ is H and R₄ is

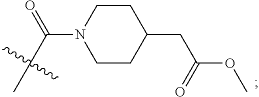 [SMILES: *C(=O)N1CCC(CC(=O)OC)CC1]

or R₂ is H, R₃ is

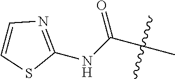 [SMILES: *C(=O)Nc1nccs1]

### and R₄ is

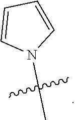 [SMILES: *n1cccc1]

When R₁ is ethyl R₂ and R₃ are H and R₄ is

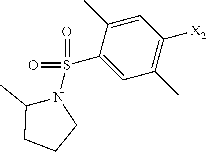

wherein X₂ is H or methyl.

In one embodiment the compound of formula I is KB-151 and has the formula

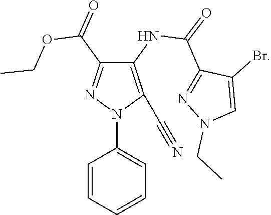 [SMILES: CCOC(=O)c1nn(-c2ccccc2)c(C#N)c1NC(=O)c1nn(CC)cc1Br]

In one embodiment, the compound of formula I can be modified to have a halogen group different than bromine, an ethyl group instead or be replaced by hydrogen. The removal of the bromine group may improve biocompatibility and reduce toxicity of the compound. In one embodiment the compound of formula I is KB-151a and has the formula

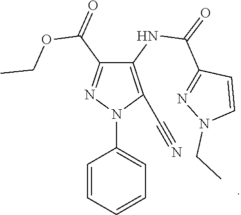 [SMILES: CCOC(=O)c1nn(-c2ccccc2)c(C#N)c1NC(=O)c1ccn(CC)n1]

In one embodiment the compound of formula I is KB-151b and has the formula

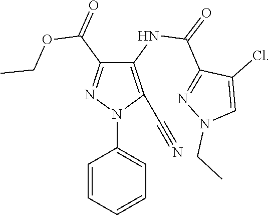 [SMILES: CCOC(=O)c1nn(-c2ccccc2)c(C#N)c1NC(=O)c1nn(CC)cc1Cl]

In one embodiment the compound of formula I is KB-151c and has the formula

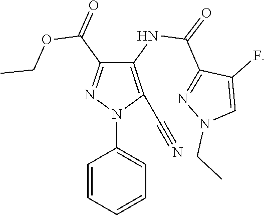 [SMILES: CCOC(=O)c1nn(-c2ccccc2)c(C#N)c1NC(=O)c1nn(CC)cc1F]

In one embodiment the compound of formula I is KB-151d and has the formula

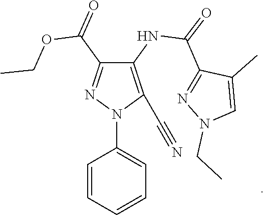 [SMILES: CCOC(=O)c1nn(-c2ccccc2)c(C#N)c1NC(=O)c1nn(CC)cc1C]

In one embodiment the compound of formula I is KB-208 and has the formula

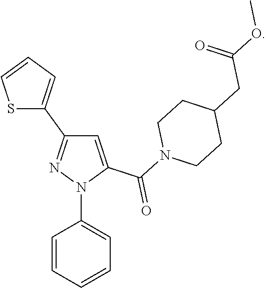 [SMILES: COC(=O)CC1CCN(C(=O)c2cc(-c3cccs3)nn2-c2ccccc2)CC1]

In one embodiment the compound of formula I is KB-198 and has the formula

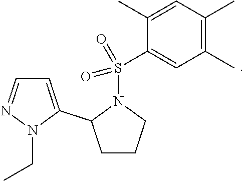 [SMILES: CCn1nccc1C1CCCN1S(=O)(=O)c1cc(C)c(C)cc1C]

In one embodiment the compound of formula I is KB-198 and has the formula

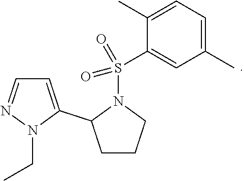 [SMILES: CCn1nccc1C1CCCN1S(=O)(=O)c1cc(C)ccc1C]

In one embodiment the compound of formula I is KB-210 and has the formula

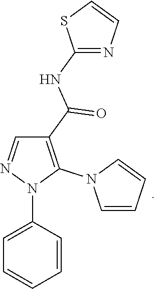 [SMILES: O=C(Nc1nccs1)c1cnn(-c2ccccc2)c1-n1cccc1]

There is provided a method of preventing, treating and/or alleviating the symptoms of phagocytic activity on immune cells in a subject in need thereof. In some embodiments, the phagocytosis is caused by a conditions in which the subject generates antibodies against certain types of hematopoietic cells in their blood, and the phagocytic cells (e.g. monocyte or macrophage) phagocytose the hematopoietic cells coated with those antibodies. One example of such conditions is an immune cytopenia. The present compounds can be used to prevent, treat and/or alleviate the symptoms of an immune cytopenia. There are different types of immune cytopenias such as (a) immune thrombocytopenia (ITP; autoimmune disease characterized by increased platelet destruction in the spleen and liver and/or decreased platelet production in the bone marrow), (b) hemolytic disease of the fetus and newborn (HDFN; maternal hemolytic antibodies crossing the placenta), (c) autoimmune hemolytic anemia (AIHA; phagocytosis of autoantibody-coated red blood cells), (d) alloimmune hemolytic anemias such as hemolytic transfusion reaction (HTR; phagocytosis of donor red blood cells due to preformed hemolytic alloantibodies to the donor red cell antigens), (e) delayed hemolytic transfusion reaction (DHTR; development of hemolytic alloantibodies following transfusion), and (f autoimmune neutropenia (AlN) associated with autoantibodies produced against neutrophils, mainly affecting children. More generally, the present disclosure provides a method for the prevention, treatment and/or alleviation of one or more autoimmune or alloimmune disease comprising administering to a subject in need thereof a therapeutically effective amount of a compound as defined herein.

Administration is by any of the routes normally used for introducing an agent into ultimate contact with blood. The agent described herein can be administered in any suitable manner, preferably with pharmaceutically acceptable carriers or excipients. The terms “pharmaceutically acceptable carrier”, “excipients”, “physiologically acceptable vehicle” and the like are to be understood as referring to an acceptable carrier that may be administered to a subject, together with the agent, and which does not destroy the pharmacological activity thereof. Further, as used herein “pharmaceutically acceptable carrier” or “pharmaceutical carrier” are known in the art and include, but are not limited to, 0.01-0.1 M and preferably 0.05 M phosphate buffer or 0.9% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

In some embodiments, there is provided a pharmaceutical composition comprising a compound of formula I (e.g. KB-151, KB-151a, KB-151b, KB-151c, KB-151d, KB-208, KB-198, KB-198a, and/or KB-210) and a pharmaceutically acceptable carrier. The pharmaceutical composition can be used in the prevention, alleviation or treatment methods described herein. As used herein, “pharmaceutical composition” means therapeutically effective amounts (dose) of the compound together with pharmaceutically acceptable diluents, preservatives, solubilizers, emulsifiers, and/or carriers. A “therapeutically effective amount” as used herein in the context of the pharmaceutical composition refers to that amount which provides a therapeutic effect for a given condition and administration regimen. Such compositions are liquids or lyophilized or otherwise dried formulations and include diluents of various buffer content (e.g., Tris-HCl, acetate, phosphate), pH and ionic strength, additives such as albumin or gelatin to prevent absorption to surfaces, and detergents (e.g., Tween 20™, Tween 80™, Pluronic F68™, bile acid salts). The pharmaceutical composition can comprise pharmaceutically acceptable solubilizing agents (e.g., glycerol, polyethylene glycerol), anti-oxidants (e.g., ascorbic acid, sodium metabisulfite), preservatives (e.g., thimerosal, benzyl alcohol, parabens), bulking substances or tonicity modifiers (e.g., lactose, mannitol), covalent attachment of polymers such as polyethylene glycol, complexation with metal ions, or incorporation of the material into or onto particulate preparations of polymeric compounds such as polylactic acid, polyglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also contemplated by the present disclosure are particulate compositions coated with polymers (e.g., poloxamers or poloxamines).

Suitable methods of administering the agent are available and well known to those of skill in the art, and, although more than one route can be used to administer a particular composition, a particular route can often provide a more immediate and more effective reaction than another route. The preventive or therapeutic compounds of the present invention may be administered, either orally or parenterally, systemically or locally. For example, intravenous injection such as drip infusion, intramuscular injection, intraperitoneal injection, subcutaneous injection, suppositories, intestinal lavage, oral enteric coated tablets, and the like can be selected, and the method of administration may be chosen, as appropriate, depending on the age and the conditions of the patient.

In some embodiments, the treatment can comprise administering to a subject in need thereof a compound according to the present disclosure in a therapeutically effective amount to inhibit phagocytosis, for example in the context of an immune cytopenia. A “therapeutically effective amount” as used herein also refers to an amount (dose) effective in inhibiting or reducing the phagocytosis of blood cells in the subject in need thereof. It is also to be understood herein that a “pharmaceutically effective amount” may be interpreted as an amount giving a desired therapeutic effect, either taken in one dose or in any dosage or route, taken alone or in combination with other therapeutic agents. In some embodiments, the compounds or pharmaceutical compositions according to the present disclosure and other therapeutic agent(s) are administered at the same time or within a predetermined time interval (ranging from a minute, an hour, a day, a week or a month for example). The therapeutic effect includes but is not limited to the prevention, treatment and/or alleviation of symptoms of immune cytopenias. More specifically, the “prevention, treatment and/or alleviation of symptoms of immune cytopenias” refer to the ability of the compound or the pharmaceutical composition to limit the development, progression and/or symptomology of the immune cytopenia. Symptoms associated with an immune thrombocytopenia include, but are not limited to: bleeding nose, bleeding gums, easy and excessive bruising, superficial bleeding into the skin, bleeding into the brain, blood in urine, blood in the stool, and increased blood loss during the menstrual period. Symptoms associated with immune hemolytic conditions include, but are not limited to: fatigue, jaundice, rigors, shortness of breath, and dizziness.

The excipient(s) or carrier(s) must be “pharmaceutically acceptable” in the sense of being compatible with the other ingredients of the formulation and not being deleterious to the recipient thereof. Standard accepted excipient(s) or carrier(s) are well known to skilled practitioners and described in numerous textbooks.

It will be clear to a person skilled in the art that the amount of the compound described herein and used in accordance with the disclosure (or if a further additional therapeutic agent is required or desired) can be determined by the attending physician or pharmacist. It will be appreciated that the amount of a compound required will vary not only with the particular compound selected but also with the route of administration, the nature of the condition for which treatment is required and the age and condition of the patient. It will be understood that the scope of the method of treatment or uses described herein is not particularly limited, but includes in principle any therapeutically useful outcome including preventing, treating or slowing the progression of conditions defined herein.

One aspect of the present work on small molecule phagocytosis inhibitors is the advantage of being a treatment that can work quickly. Indeed, treatments that can rapidly reverse the immune-mediated destruction of specific blood cells would be highly beneficial; in general, and specifically, they would provide additional time for implementing other therapeutic strategies and a better chance for these strategies to be successful. Hence, the present compounds can be more specific and have a broader application than the current treatment options of immune cytopenias and may be used in addition or in conjunction with existing treatment options. For example, these inhibitors could be administered in an emergency setting as sole therapeutics or co-administered with other ITP treatment options.

The compounds of the present disclosure were also observed to decrease the level of low density lipoprotein (LDL) and triglycerides. Therefore, the compounds of the present disclosure, particularly KB-151 and KB-208 are useful for reducing the LDL and triglycerides in an individual in need thereof.

The mechanism of action of KB-151 and KB-208 was determined to be the dephosphorylation of HSP27. HSP27 is a multidimensional protein that acts as a protein chaperone. Therefore, the benefit of KB-151 and KB-208 extends beyond immune cytopenias and to other conditions where HSP27 is deregulated (i.e. increased phosphorylation). HSP27 is known to have a role in cardiovascular disease for example atherosclerosis. In one embodiment, KB-151 and/or KB-208 are used in the treatment, prevention or alleviation of symptoms of atherosclerosis. HSP27 also plays a role in the inhibition of apoptosis and actin cytoskeletal remodeling. Overexpression of HSP27 leads to an increase in the concentration of filamentous actin (F-actin) at the cell cortex as well as an increase in pinocytotic activity. Overexpression of the non-phosphorylatable mutant form of HSP27 decreases cortical F-actin concentration and pinocytosis activity relative to control cells. Without wishing to be bound by theory, this can explain why the inhibition of HSP27 phosphorylation can result in phagocytosis inhibition. Due to its role in apoptosis, HSP27 is also involved in certain cancers such as prostate cancer, colorectal cancer and breast cancer. There is thus also provided methods of treating, preventing or alleviating the symptoms of a cancer with an increase in HSP27 phosphorylation with the compound of formula I (e.g. KB-151, KB-151a, KB-151 b, KB-151c, KB-151d, KB-208, KB-198, KB-198a, and/or KB-210).

### EXAMPLE

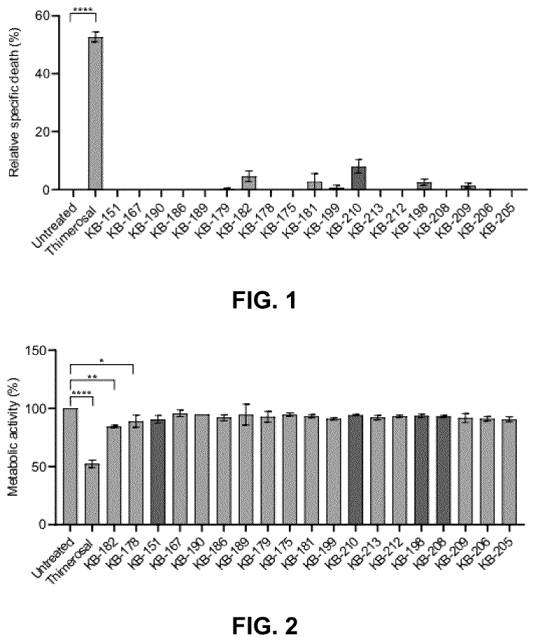

A first filter screen of over 13282 compounds was conducted to explore small molecule agents that inhibit phagocytosis. The screening was conducted using the following parameters (1) a molecular weight of 200-500 Da, (2) a calculated partition coefficient (cLogP)≤4, (3) having less than 4 chiral centers, (4) having 3 or more heteroatoms, (5) not being a natural product/compound. A diversity score (Tanimoto coefficient) of 0.65 was selected. The results of this first screen led to the acquisition of a 5000 compound library, which was further narrowed based on having at least one pyrazole or pyrrole group. These incorporated backbones may be capable of favourably interacting with the reactive portions in the cell surface of the macrophage. From this second filter screen, 80 compounds were selected for a preliminary in vitro iterative exploration. A third round of filters was applied, whereby compounds that showed at least 69% inhibition in the monocyte monolayer assay (MMA), were further investigated in vitro (Tables 1-2 and FIG. 1). From the molecules of Table 2, two lead compounds (KB-151 and KB-208) have been selected having negligible toxicity, tested by MTT ((3-(4,5-dimethylthiazol-2-yl)-2,5-diphenyltetrazolium bromide) tetrazolium) viability, lactate dehydrogenase (LDH) release and apoptosis, and high efficacy. The IC₅₀ tested using an in vitro phagocytosis assay for inhibition of phagocytosis was from about 2 to about 4 μM.

TABLE 1
Screening of the 80 compounds

| | | Average |
|---|---|---|
| | | inhibition |
| | | (% ± |
| | | standard |
| Compound | Structure | deviation) |
| KB-132 | 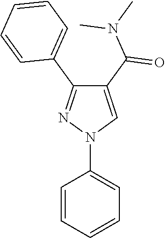 [SMILES: CN(C)C(=O)c1cn(-c2ccccc2)nc1-c1ccccc1] | −9 ± 3.47 |
| KB-133 | 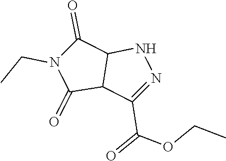 [SMILES: CCOC(=O)C1=NNC2C(=O)N(CC)C(=O)C12] | 24 ± 8.98 |
| KB-134 | 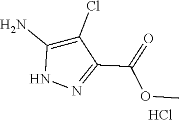 | 9 ± 6.99 |
| KB-135 | 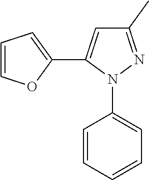 [SMILES: Cc1cc(-c2ccco2)n(-c2ccccc2)n1] | 18 ± 2.7 |
| KB-136 | 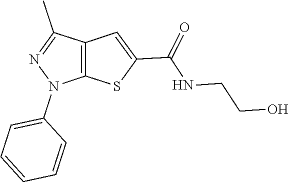 [SMILES: Cc1nn(-c2ccccc2)c2sc(C(=O)NCCO)cc12] | −4 ± 1.82 |
| KB-137 | 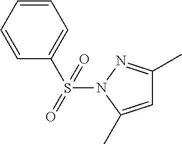 [SMILES: Cc1cc(C)n(S(=O)(=O)c2ccccc2)n1] | 10 ± 1.21 |
| KB-138 | 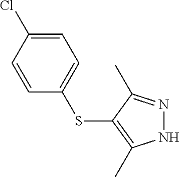 | 38 ± 6.56 |
| KB-139 | 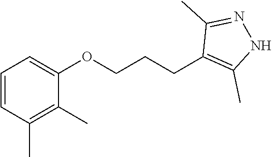 [SMILES: Cc1cccc(OCCCc2c(C)n[nH]c2C)c1C] | 6 ± 3.48 |
| KB-140 | 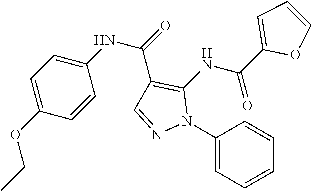 [SMILES: CCOc1ccc(NC(=O)c2cnn(-c3ccccc3)c2NC(=O)c2ccco2)cc1] | 31 ± 7.84 |
| KB-141 | 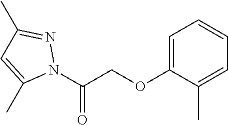 [SMILES: Cc1cc(C)n(C(=O)COc2ccccc2C)n1] | 36 ± 9.37 |
| KB-142 | 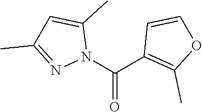 | 11 ± 6.51 |
| KB-143 | 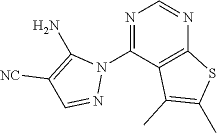 [SMILES: Cc1sc2ncnc(-n3ncc(C#N)c3N)c2c1C] | 10 ± 6.87 |
| KB-144 | 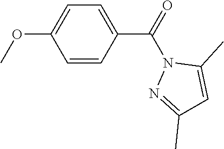 | 33 ± 1.10 |
| KB-145 | 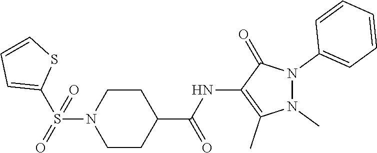 [SMILES: Cc1c(NC(=O)C2CCN(S(=O)(=O)c3cccs3)CC2)c(=O)n(-c2ccccc2)n1C] | 4 ± 0.06 |
| KB-146 | 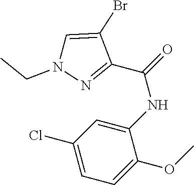 [SMILES: CCn1cc(Br)c(C(=O)Nc2cc(Cl)ccc2OC)n1] | 10 ± 9.56 |
| KB-147 | 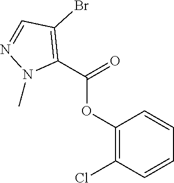 [SMILES: Cn1ncc(Br)c1C(=O)Oc1ccccc1Cl] | 0 ± 3.16 |
| KB-148 | 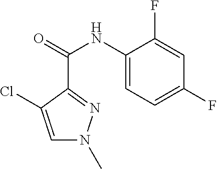 [SMILES: Cn1cc(Cl)c(C(=O)Nc2ccc(F)cc2F)n1] | 28 ± 9.62 |
| KB-149 | 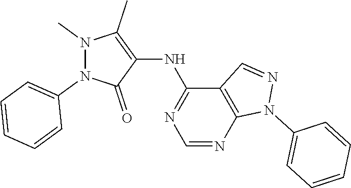 [SMILES: Cc1c(Nc2ncnc3c2cnn3-c2ccccc2)c(=O)n(-c2ccccc2)n1C] | 0 ± 2.56 |
| KB-150 | 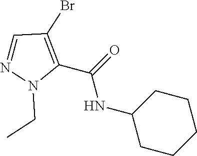 [SMILES: CCn1ncc(Br)c1C(=O)NC1CCCCC1] | 24 ± 8.58 |
| KB-151 | 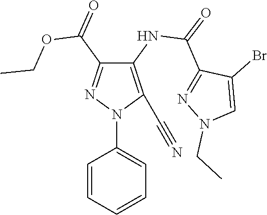 [SMILES: CCOC(=O)c1nn(-c2ccccc2)c(C#N)c1NC(=O)c1nn(CC)cc1Br] | 80 ± 3.68 |
| KB-152 | 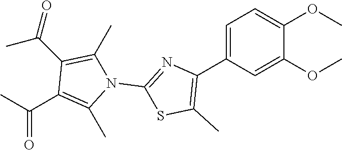 [SMILES: COc1ccc(-c2nc(-n3c(C)c(C(C)=O)c(C(C)=O)c3C)sc2C)cc1OC] | 2 ± 8.84 |
| KB-153 | 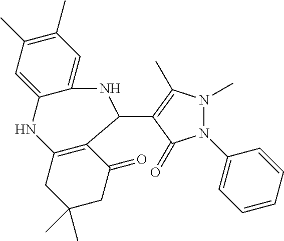 [SMILES: Cc1cc2c(cc1C)NC(c1c(C)n(C)n(-c3ccccc3)c1=O)C1=C(CC(C)(C)CC1=O)N2] | 8 ± 8.3 |
| KB-154 | 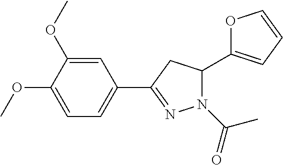 [SMILES: COc1ccc(C2=NN(C(C)=O)C(c3ccco3)C2)cc1OC] | 2 ± 5.19 |
| KB-155 | 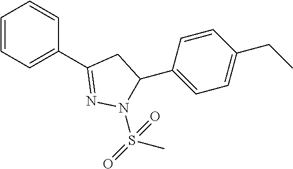 [SMILES: CCc1ccc(C2CC(c3ccccc3)=NN2S(C)(=O)=O)cc1] | 8 ± 9.35 |
| KB-156 | 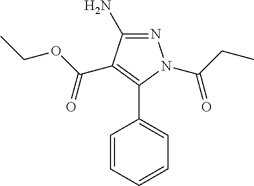 [SMILES: CCOC(=O)c1c(N)nn(C(=O)CC)c1-c1ccccc1] | 2 ± 6.64 |
| KB-157 | 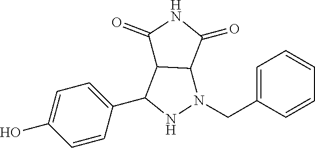 [SMILES: O=C1NC(=O)C2C1C(c1ccc(O)cc1)NN2Cc1ccccc1] | 3 ± 9.96 |
| KB-158 | 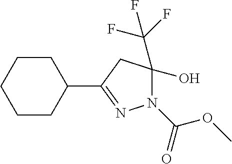 [SMILES: COC(=O)N1N=C(C2CCCCC2)CC1(O)C(F)(F)F] | 0 ± 9.11 |
| KB-159 | 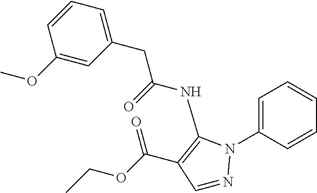 [SMILES: CCOC(=O)c1cnn(-c2ccccc2)c1NC(=O)Cc1cccc(OC)c1] | 15 ± 8.4 |
| KB-160 | 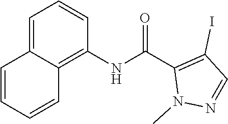 [SMILES: Cn1ncc(I)c1C(=O)Nc1cccc2ccccc12] | −2 ± 5.32 |
| KB-161 | 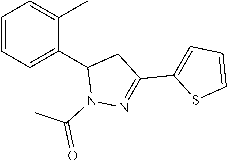 [SMILES: CC(=O)N1N=C(c2cccs2)CC1c1ccccc1C] | 7 ± 8.43 |
| KB-162 | 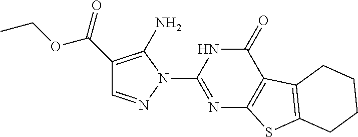 [SMILES: CCOC(=O)c1cnn(-c2nc3sc4c(c3c(=O)[nH]2)CCCC4)c1N] | −37 ± 2.59 |
| KB-163 | 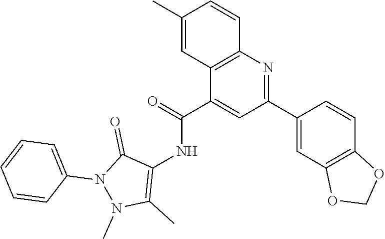 [SMILES: Cc1ccc2nc(-c3ccc4c(c3)OCO4)cc(C(=O)Nc3c(C)n(C)n(-c4ccccc4)c3=O)c2c1] | −30 ± 1.77 |
| KB-164 | 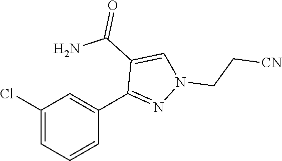 [SMILES: N#CCCn1cc(C(N)=O)c(-c2cccc(Cl)c2)n1] | −3 ± 9.7 |
| KB-165 | 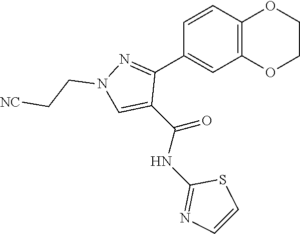 [SMILES: N#CCCn1cc(C(=O)Nc2nccs2)c(-c2ccc3c(c2)OCCO3)n1] | 33 ± 5.3 |
| KB-166 | 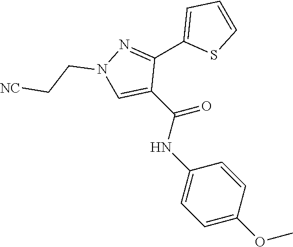 [SMILES: COc1ccc(NC(=O)c2cn(CCC#N)nc2-c2cccs2)cc1] | 17 ± 3.91 |
| KB-167 | 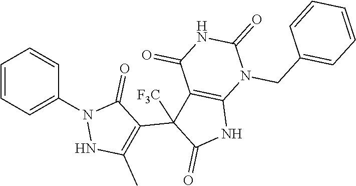 [SMILES: Cc1[nH]n(-c2ccccc2)c(=O)c1C1(C(F)(F)F)C(=O)Nc2c1c(=O)[nH]c(=O)n2Cc1ccccc1] | 53 ± 4.18 |
| KB-168 | 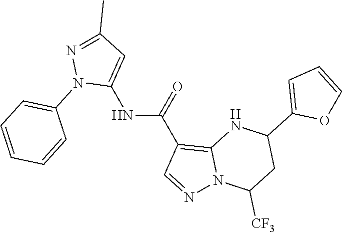 [SMILES: Cc1cc(NC(=O)c2cnn3c2NC(c2ccco2)CC3C(F)(F)F)n(-c2ccccc2)n1] | 23 ± 3.86 |
| KB-169 | 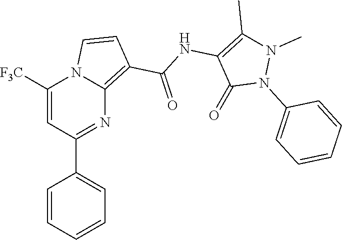 [SMILES: Cc1c(NC(=O)c2ccn3c(C(F)(F)F)cc(-c4ccccc4)nc23)c(=O)n(-c2ccccc2)n1C] | 0 ± 5.57 |
| KB-170 | 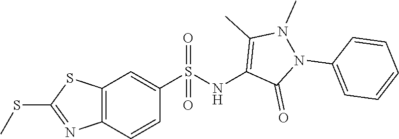 [SMILES: CSc1nc2ccc(S(=O)(=O)Nc3c(C)n(C)n(-c4ccccc4)c3=O)cc2s1] | −13 ± 4.31 |
| KB-171 | 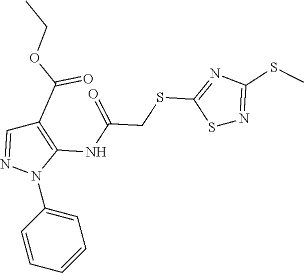 [SMILES: CCOC(=O)c1cnn(-c2ccccc2)c1NC(=O)CSc1nc(SC)ns1] | 7 ± 0.39 |
| KB-173 | 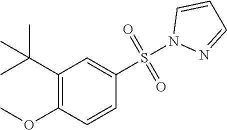 [SMILES: COc1ccc(S(=O)(=O)n2cccn2)cc1C(C)(C)C] | −8 ± 8.88 |
| KB-174 | 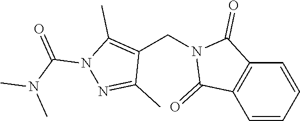 [SMILES: Cc1nn(C(=O)N(C)C)c(C)c1CN1C(=O)c2ccccc2C1=O] | 39 ± 9.53 |
| KB-175 | 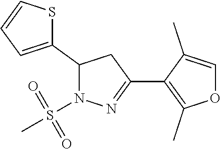 [SMILES: Cc1coc(C)c1C1=NN(S(C)(=O)=O)C(c2cccs2)C1] | 50 ± 1.45 |
| KB-176 | 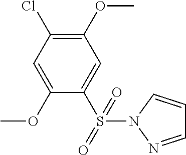 [SMILES: COc1cc(S(=O)(=O)n2cccn2)c(OC)cc1Cl] | −10 ± 7.06 |
| KB-177 | 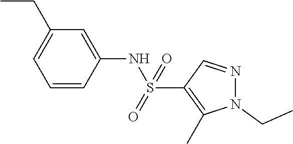 [SMILES: CCc1cccc(NS(=O)(=O)c2cnn(CC)c2C)c1] | 4 ± 9.76 |
| KB-178 | 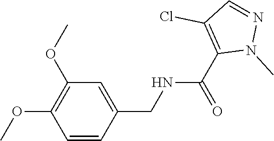 | 53 ± 5.11 |
| KB-179 | 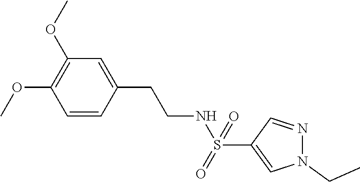 [SMILES: CCn1cc(S(=O)(=O)NCCc2ccc(OC)c(OC)c2)cn1] | 53 ± 1.18 |
| KB-180 | 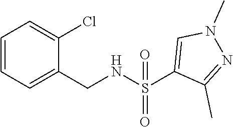 [SMILES: Cc1nn(C)cc1S(=O)(=O)NCc1ccccc1Cl] | −42 ± 8.81 |
| KB-181 | 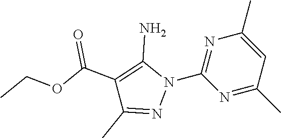 [SMILES: CCOC(=O)c1c(C)nn(-c2nc(C)cc(C)n2)c1N] | −11 ± 10.27 |
| KB-182 | 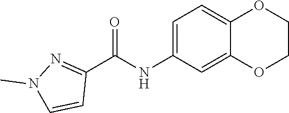 [SMILES: Cn1ccc(C(=O)Nc2ccc3c(c2)OCCO3)n1] | 55 ± 3.65 |
| KB-183 | 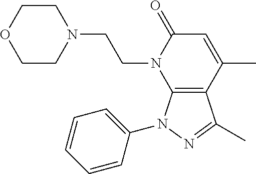 [SMILES: Cc1cc(=O)n(CCN2CCOCC2)c2c1c(C)nn2-c1ccccc1] | −28 ± 9.96 |
| KB-184 | 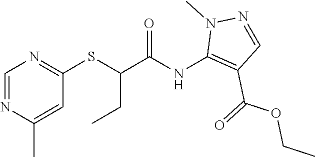 [SMILES: CCOC(=O)c1cnn(C)c1NC(=O)C(CC)Sc1cc(C)ncn1] | 7 ± 2.92 |
| KB-185 | 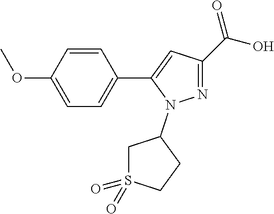 [SMILES: COc1ccc(-c2cc(C(=O)O)nn2C2CCS(=O)(=O)C2)cc1] | −3 ± 3.6 |
| KB-186 | 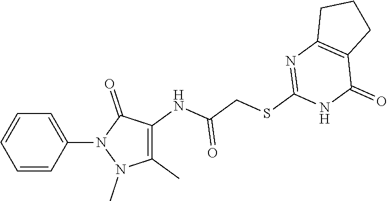 [SMILES: Cc1c(NC(=O)CSc2nc3c(c(=O)[nH]2)CCC3)c(=O)n(-c2ccccc2)n1C] | 55 ± 8.43 |
| KB-187 | 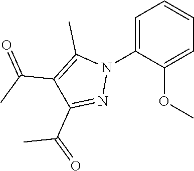 [SMILES: COc1ccccc1-n1nc(C(C)=O)c(C(C)=O)c1C] | −16 ± 9.33 |
| KB-188 | 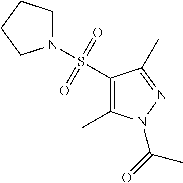 [SMILES: CC(=O)n1nc(C)c(S(=O)(=O)N2CCCC2)c1C] | −26 ± 7.94 |
| KB-189 | 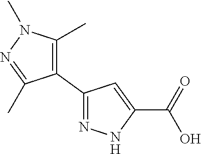 [SMILES: Cc1nn(C)c(C)c1-c1cc(C(=O)O)[nH]n1] | 52 ± 4.75 |
| KB-190 | 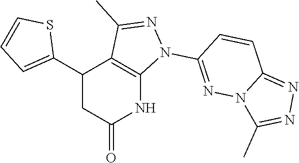 [SMILES: Cc1nn(-c2ccc3nnc(C)n3n2)c2c1C(c1cccs1)CC(=O)N2] | 56 ± 1.24 |
| KB-191 | 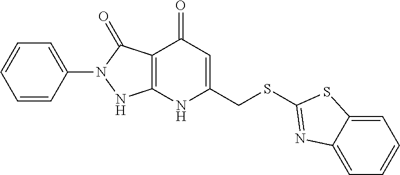 [SMILES: O=c1cc(CSc2nc3ccccc3s2)[nH]c2[nH]n(-c3ccccc3)c(=O)c12] | −23 ± 7.85 |
| KB-192 | 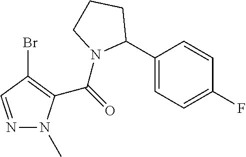 [SMILES: Cn1ncc(Br)c1C(=O)N1CCCC1c1ccc(F)cc1] | −29 ± 9.54 |
| KB-194 | 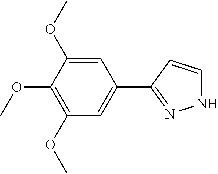 [SMILES: COc1cc(-c2cc[nH]n2)cc(OC)c1OC] | 39 ± 4.96 |
| KB-195 | 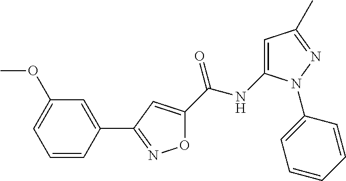 [SMILES: COc1cccc(-c2cc(C(=O)Nc3cc(C)nn3-c3ccccc3)on2)c1] | 25 ± 6.36 |
| KB-196 | 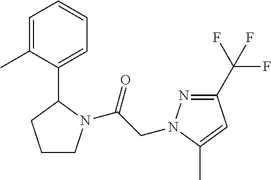 [SMILES: Cc1ccccc1C1CCCN1C(=O)Cn1nc(C(F)(F)F)cc1C] | 24 ± 5.76 |
| KB-197 | 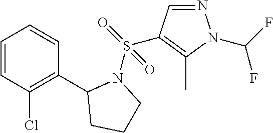 [SMILES: Cc1c(S(=O)(=O)N2CCCC2c2ccccc2Cl)cnn1C(F)F] | 45 ± 4.97 |
| KB-198 | 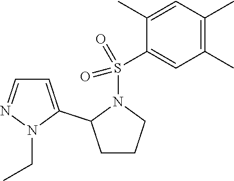 [SMILES: CCn1nccc1C1CCCN1S(=O)(=O)c1cc(C)c(C)cc1C] | 75 ± 4.76 |
| KB-199 | 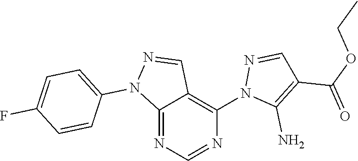 [SMILES: CCOC(=O)c1cnn(-c2ncnc3c2cnn3-c2ccc(F)cc2)c1N] | 40 ± 5.46 |
| KB-200 | 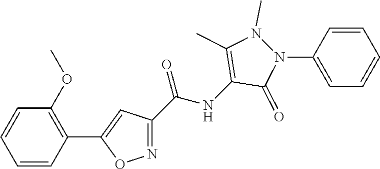 [SMILES: COc1ccccc1-c1cc(C(=O)Nc2c(C)n(C)n(-c3ccccc3)c2=O)no1] | 31 ± 1.84 |
| KB-201 | 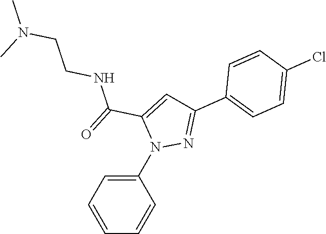 [SMILES: CN(C)CCNC(=O)c1cc(-c2ccc(Cl)cc2)nn1-c1ccccc1] | 0 ± 5.41 |
| KB-202 | 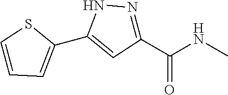 | 33 ± 2.31 |
| KB-203 | 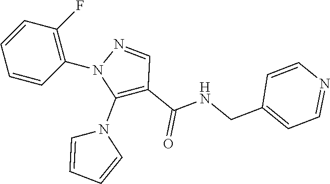 [SMILES: O=C(NCc1ccncc1)c1cnn(-c2ccccc2F)c1-n1cccc1] | 27 ± 2.72 |
| KB-204 | 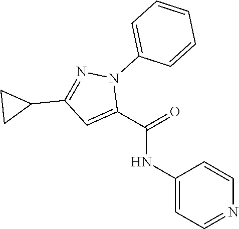 [SMILES: O=C(Nc1ccncc1)c1cc(C2CC2)nn1-c1ccccc1] | 36 ± 3.6 |
| KB-205 | 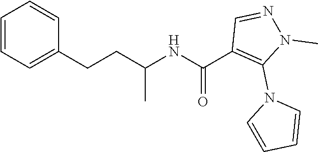 [SMILES: CC(CCc1ccccc1)NC(=O)c1cnn(C)c1-n1cccc1] | 47 ± 0.98 |
| KB-206 | 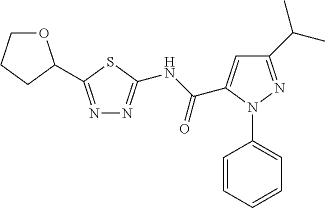 [SMILES: CC(C)c1cc(C(=O)Nc2nnc(C3CCCO3)s2)n(-c2ccccc2)n1] | 50 ± 5.46 |
| KB-207 | 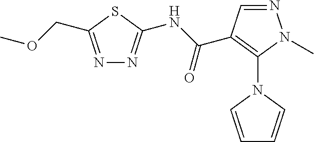 | 14 ± 1.86 |
| KB-208 | 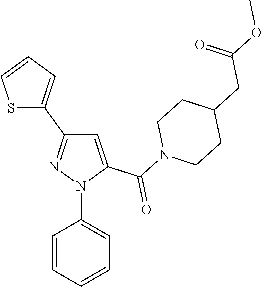 [SMILES: COC(=O)CC1CCN(C(=O)c2cc(-c3cccs3)nn2-c2ccccc2)CC1] | 81 ± 3.43 |
| KB-209 | 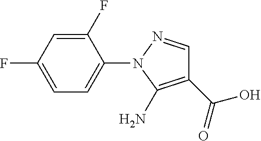 [SMILES: Nc1c(C(=O)O)cnn1-c1ccc(F)cc1F] | 50 ± 1.02 |
| KB-210 | 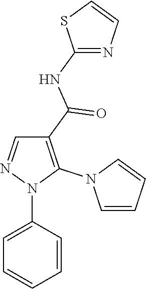 [SMILES: O=C(Nc1nccs1)c1cnn(-c2ccccc2)c1-n1cccc1] | 69 ± 5.32 |
| KB-211 | 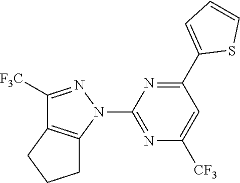 [SMILES: FC(F)(F)c1cc(-c2cccs2)nc(-n2nc(C(F)(F)F)c3c2CCC3)n1] | 33 ± 2.31 |
| KB-212 | 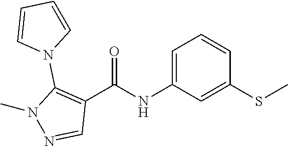 | 47 ± 9.67 |
| KB-213 | 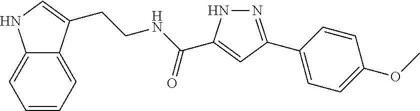 [SMILES: COc1ccc(-c2cc(C(=O)NCCc3c[nH]c4ccccc34)[nH]n2)cc1] | 64 ± 8.74 |

TABLE 2
Compounds that inhibited anti-D-opsonized Rh(D+)
by ≥40% phagocytic inhibition

| Compound | Inhibition percentage | Phagocytic index (PI) |
|---|---|---|
| KB-151 | 80 | 17 |
| KB-167 | 53 | 14 |
| KB-175 | 50 | 15 |
| KB-178 | 53 | 29 |
| KB-179 | 53 | 14 |
| KB-181 | 45 | 37 |
| KB-182 | 55 | 28 |
| KB-186 | 55 | 28 |
| KB-189 | 52 | 30 |
| KB-190 | 56 | 27 |
| KB-198 | 75 | 9 |
| KB-199 | 40 | 40 |
| KB-205 | 47 | 19 |
| KB-206 | 50 | 18 |
| KB-208 | 81 | 7 |
| KB-209 | 50 | 18 |
| KB-210 | 69 | 11 |
| KB-212 | 47 | 19 |
| KB-213 | 64 | 13 |

PI = (# of phagocytic red blood cells)/(300 monocytes) * 100

Upon informed consent, blood from healthy volunteers was collected in acid-citrate-dextrose (ACD) anticoagulant-containing tubes. Mononuclear cells (PBMC) were obtained by density centrifugation using Ficoll-Hypaque solution (Biochrom AG). Primary monocytes for functional phagocytosis assays were separated as described below.

Cell lines (HepG2, liver-derived, HB-8065™; HEK293, kidney-derived cell line, CRL-1573™) were purchased from the ATCC. Both cell lines were cultured in Eagle's Minimum Essential Medium supplemented with 10% fetal calf serum.

The MMA to screen compounds for their ability to inhibit phagocytosis was performed as previously described (Purohit, M. K. et al., Bioorg. Med. Chem. Lett. 2013; 23:2324-7; and Purohit, M. K. et al., Bioorg. Med. Chem. 2014; 22:2739-52.). Briefly, Mononuclear cells (PBMCs) were layered onto chamber slides and monocytes purified by adherence after 1 hr at 37° C. with 5% CO₂. Drugs were solubilized in 100% DMSO and then diluted in Roswell Park Memorial Institute (RPMI) to the final concentration of 5 μM, as previously reported (8,9). Corresponding dimethyl sulfoxide (DMSO) volumes in RPMI medium were used as a control. After one hour, indicator Rh-positive red cells opsonized with anti-RhD were added to the chamber slides and then further incubated for 2 hours before fixing and counting phagocytosed red blood cells (RBCs) using phase-contrast microscopy. As a positive control for inhibition, Intravenous immunoglobulin (IVIg) was used, known to inhibit phagocytosis due to blocking of FcγRs. The phagocytosis index (PI) was determined as the number of phagocytosed RBCs in 100 monocytes. The percent inhibition of phagocytosis was determined by comparing the PI in each treated sample to the PI in the untreated sample, which was determined by normalizing the PI concentration in each treated sample (phagocytosis of opsonized RBCs with vehicle only, representing 100 percent phagocytosis.

$$( PI = \frac{\# of phagocyt RBCs}{300 monocytes} \times 100 )$$

MTT was used to assess the impact of the selected compounds on the metabolic activity of primary mononuclear cells. PBMCs (5×10⁴ total cells per well) were isolated from ACD tubes and seeded on 96 culture plates (VWR™ Tissue Culture Plate, Untreated, Sterilized, Non-Pyrogenic). Cells were treated with 5 μM of the solubilized compounds and incubated for three hours at 37° C. with 5% CO₂. After that, MTT was added to the cells and plates were incubated for two hours. The formation of formazan purple crystals was followed under a light microscope. The formazan crystals were solubilized by incubating overnight with 100 μL of 10% sodium dodecyl sulphate (SDS), 0.01 M HCl SDS. Absorbance was measured at 570 nm (reference 690 nm) in a microplate reader (Bio-Rad™ EPOCH II). As a positive control for decreased metabolic activity, 100 μM thimerosal (diluted in RPMI) was used. Untreated cells represented the basal metabolic activity of the cells. This experiment was repeated at increasing concentrations (250 μM, 100 μM, 50 μM, and 10 μM) for two compounds (KB-151 and KB-208) using a variety of cell types, including PBMC, HEPG2, and HEK-293.

To understand if any of the studied compounds was inducing primary cell death (PBMC), lactate dehydrogenase (LDH) release was evaluated as a measure of cytotoxicity. To this end, an LDH-cytotoxicity test kit (Sigma™ Inc.) was used according to the manufacturer's instructions. Sample treatment was performed identically to the MTT assay previously described. The absorbance at 490 and 600 nm was determined using a microplate reader (Bio-Rad™ EPOCH II). Minimum lysis control was referred to as samples without treatment. Cells solubilized with a lysis buffer provided with the kit were considered as maximum lysis control. Specific cell death was calculated as: % specific death=(experimental lysis−minimum lysis)/(maximum lysis−minimum lysis)*100. This experiment was repeated at increasing concentrations (250 μM, 100 μM, 50 μM, and 10 μM) for two compounds (KB-151 and KB-208) using a variety of cell types, including PBMC, HEPG2, and HEK-293.

PBMC, HEPG2, and HEK-293 cells were treated for 1 hour with the compound KB-151 or the compound KB-208 at a 250 μM concentration. 100 μM thimerosal was used as a positive control for viability (diluted in RPMI). Staining with Annexin V and PI was performed according to the manufacturer's procedure for the Annexin-V-FLUOS Staining Kit (Sigma) and samples were run through an SP6800 Spectral Cytometer.

The MMA as explained in detail above was repeated with different concentrations of the four compounds (KB-151, KB-208, KB-198, KB-210). Concentrations included 100 μM, 50 μM, 25 μM, 10 μM, 5 μM, 2.5 μM, 1 μM, and 0.5 μM.

Statistical analysis were performed using Graphpad™ Prism 8. Student's t-test was used. P-values<0.05 were considered significant.

The 80 selected pyrazole core compounds were screened using a concentration of 5 μM in the in vitro phagocytosis assay, referred to as the monocyte monolayer assay (MMA). Compounds were compared to a standard intravenous immunoglobulin (IVIg) at a concentration of 1 mg/mL. Following this first screening, 19 compounds were identified that inhibited phagocytosis of anti-D-opsonized Rh(D+) red cells by more than 40%.

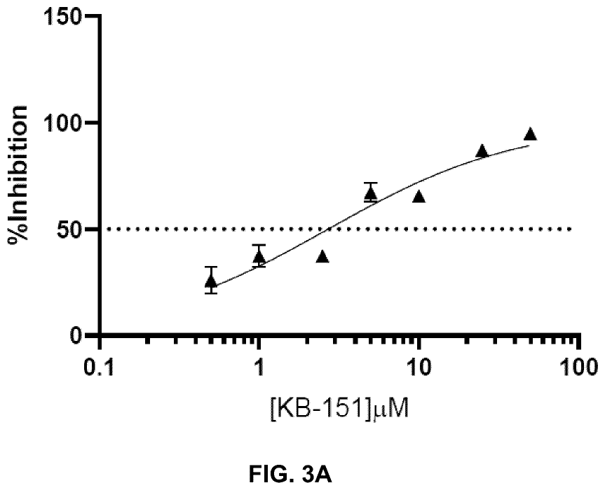

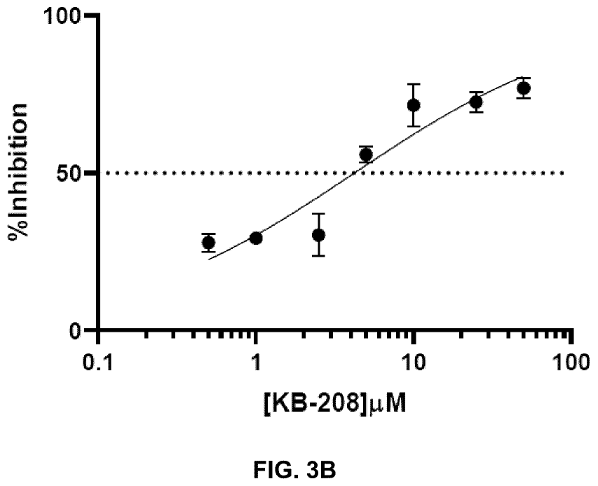

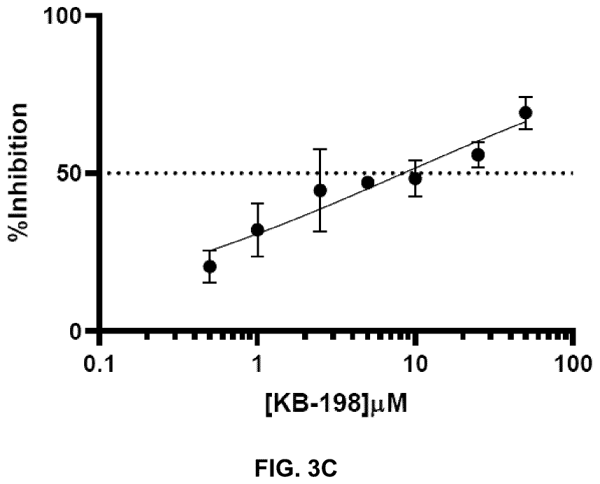

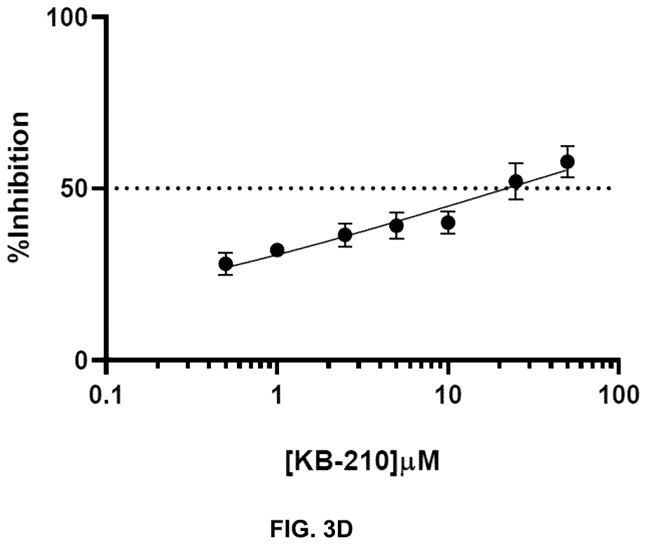

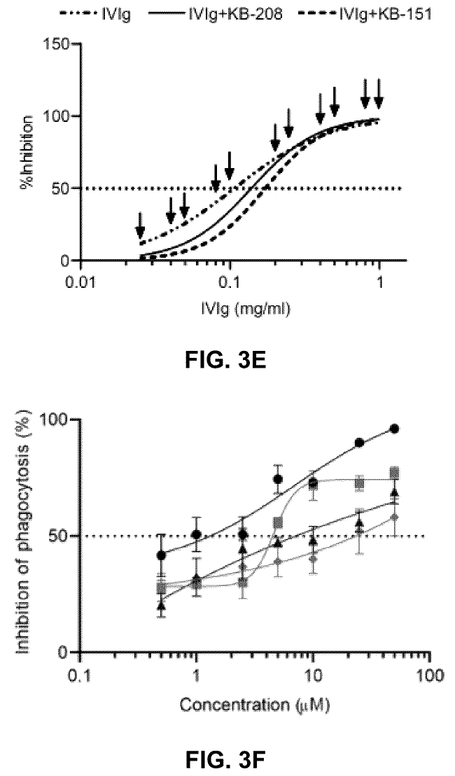

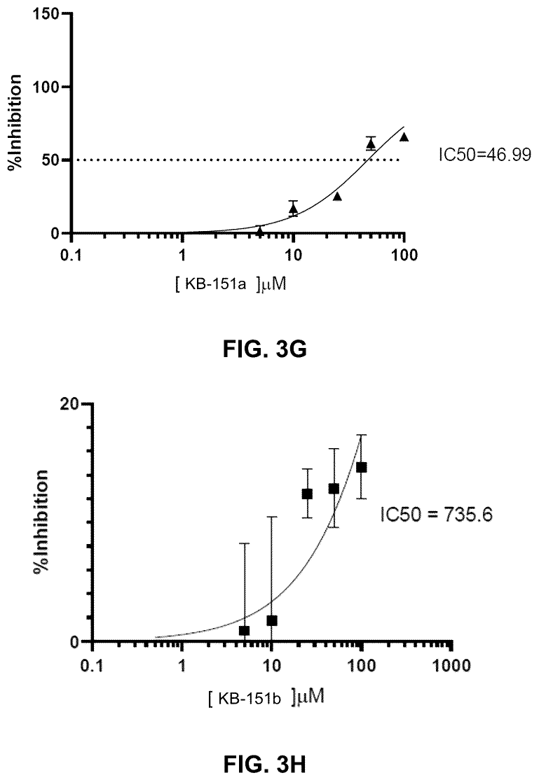

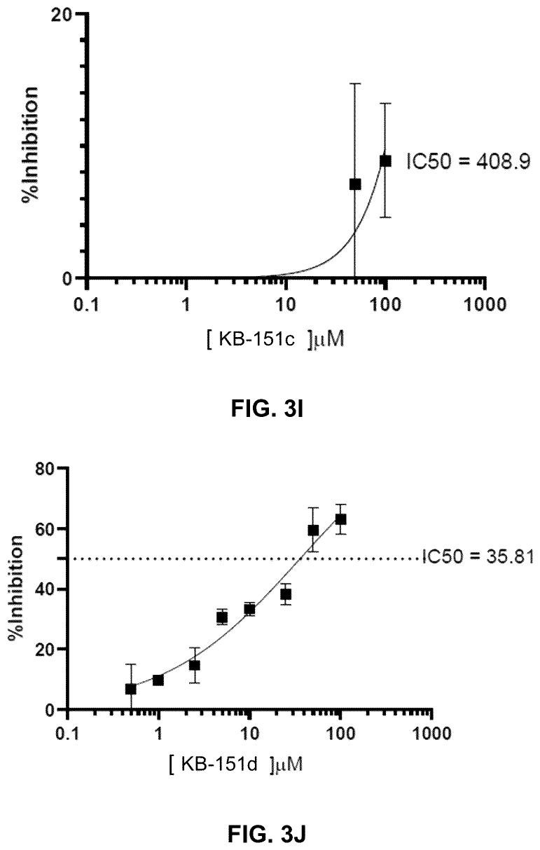

Additionally, these compounds were tested for viability/toxicity using an LDH release assay for a weakened cell membrane and the MTT assay for cell viability (FIGS. 1 and 2). Both are colorimetric assays, but LDH relies on the release of LDH enzymes into the culture medium after cell membrane disruption. As a result, the production of color suggests cytolysis. The compounds from the library were named KB-###. KB-181, 182, 198, 199, 209, and 210 demonstrated a small amount of toxicity in these experiments compared to the control, thimerosal (100 μM), but it was not significant when compared to the untreated control. None of the other chemicals released any LDH enzyme into the culture medium (FIG. 1). The MTT test is a metabolic activity assay that is based on the enzymatic conversion of MTT in viable mitochondria. The color formation is an indicator of cell viability. Thimerosal at 100 μM (****P≤0.0001), KB-182 (**P≤0.005), and KB-178 (*P≤0.05) all demonstrate statistically significant changes when compared to the untreated control (tested by Kruskal-Wallis test). Other compounds show no statistically significant change from the untreated control (FIG. 2). After reviewing the in vitro inhibition assay and the toxicity assay findings (FIG. 1 and FIG. 2), four compounds were chosen that inhibited phagocytosis by more than 65 percent (from Table 1) and that showed no toxicity in the LDH and MTT assays for further investigation. These compounds included KB-151, KB-198, KB-208, and KB-210. The dose-inhibitory response of these four drugs was determined, and the IC₅₀ values were calculated (FIGS. 3A, 3B, 3C, and 3D). IC₅₀ values for KB-210 and KB-198 were more than 5 μM (22.06 and 8.426 respectively); IC₅₀ values for KB-208 were 4.209±1.235 μM; and for KB-151, the IC₅₀ value was 2.71±0.786 μM. KB-151 and KB-208, having negligible toxicity and the lowest IC₅₀ values were then selected for further testing. KB-210 and KB-198 had an EC₅₀>10 μM. KB-208 showed an EC₅₀ of 5±0.2 μM. KB-151 showed an EC₅₀ of 8±4.

It was also tested whether or not either of these lead compounds could cooperate with IVIg in a dose-inhibitory assay whereby phagocytosis inhibition was titrated with IVIg alone and with adding each of the two compounds (KB-151 and KB-208) at their IC₅₀ concentration with the IVIg (IVIg+KB-151 or KB-208). This was done to see if the IVIg alone titration curve would shift, indicating some cooperation (synergy) with the compound(s). The compounds had no effect on the IVIg dose-response curve (FIG. 3E). Compounds KB-198 and KB-210 were also evaluated and compared with KB-151 and KB-208 (FIG. 3F). Similarly, KB-151 derivatives KB-151a, KB-151b, KB-151c and KB-151d were also evaluated (FIGS. 3G-3H). The IC₅₀ for the derivative KB-198a was found to be 226.8 μM.

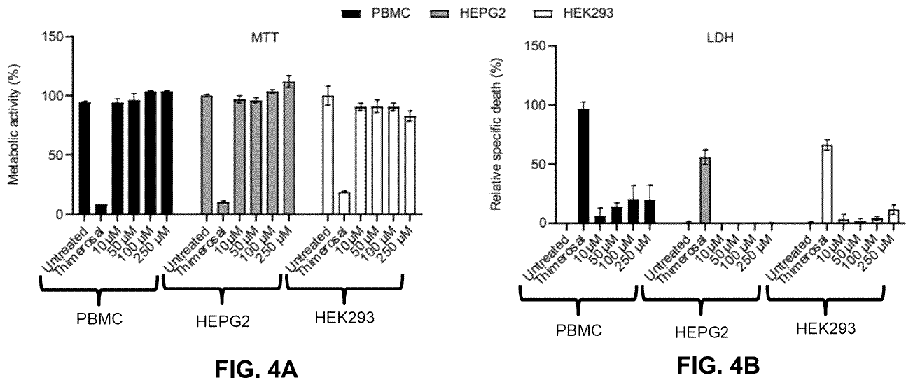

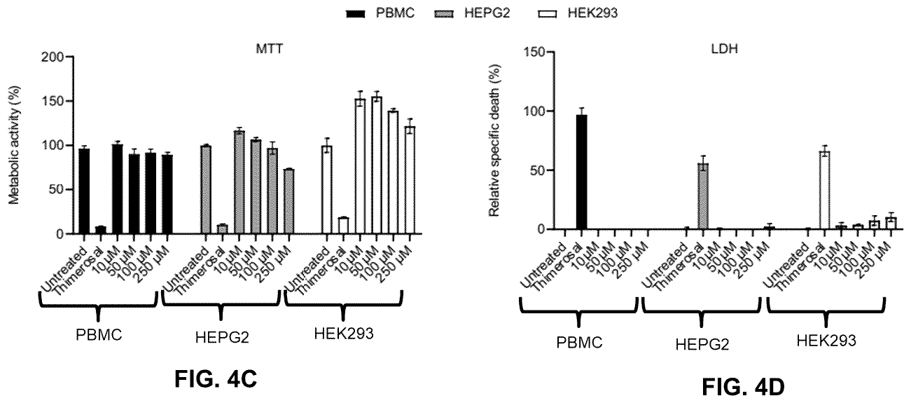

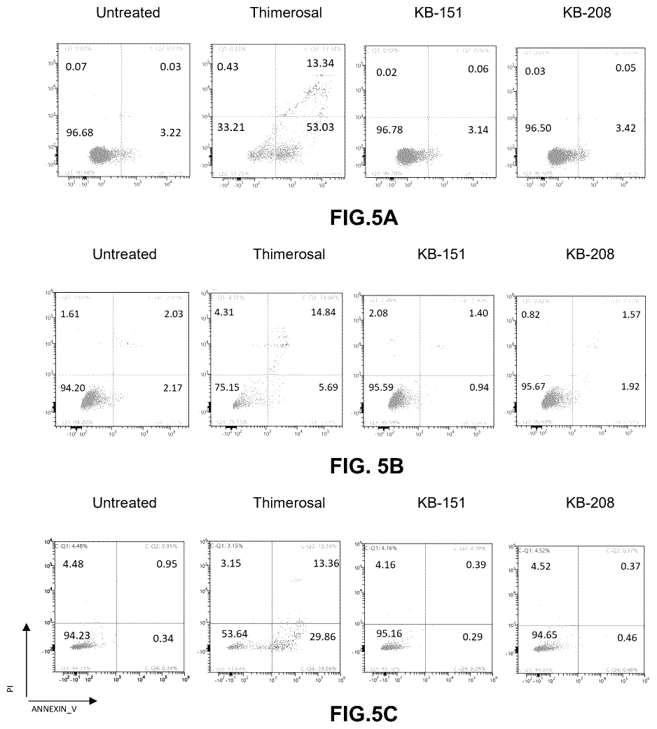

To further determine the potential for toxicity of compounds KB-151 and KB-208, additional toxicity studies were done using higher concentrations, up to 250 μM of each compound, using LDH and MTT (FIGS. 4A-4D), and an apoptosis assay (Annexin V/PI) (FIGS. 5A-5C). All assays showed low to no toxicity using peripheral blood mononuclear cells (PBMCs), liver HEPG2, and kidney HEK293 cell lines. KB-208 and KB-151 showed the same patterns in all samples, including PBMC, HEPG2, HEK-293, and similar to untreated cells.

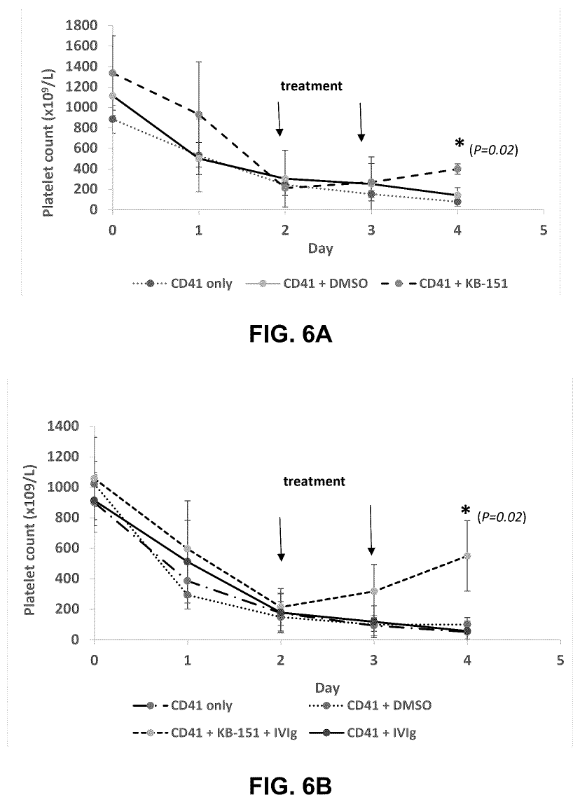

An in vivo phagocytosis inhibition assay was performed with KB-151 in a mouse model, Balb/c animals (FIG. 6A and FIG. 6B). A dose of 100 μM of KB-151 was injected to 20.5 g mice at 500 μL based on the following calculations:

$$0.0001 M \times 457.28 g / mol ( MW of KB - 151 ) = 0.045729 g / L$$

$$0.045729 g / L \times 0.5 mL ( amount injected ) = 0.0228645 mg = 22.8645 μg$$

Therefore 0.0228645 mg of KB-151 was injected to 20.5 mg mice at 0.0228645 mg/0.0205 kg=1.115 mg/kg.

A dose-escalation anti-CD41 antibody ITP mouse model was used. KB-151 showed a significant increase of the platelet (PLT) count on day 4 after two treatment doses on days 2 and 3 compared to the CD-41 group (*P≤0.05).

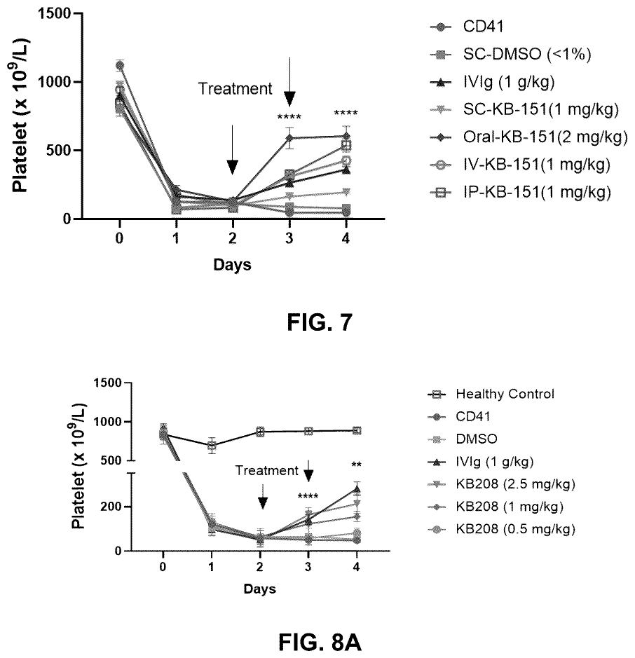

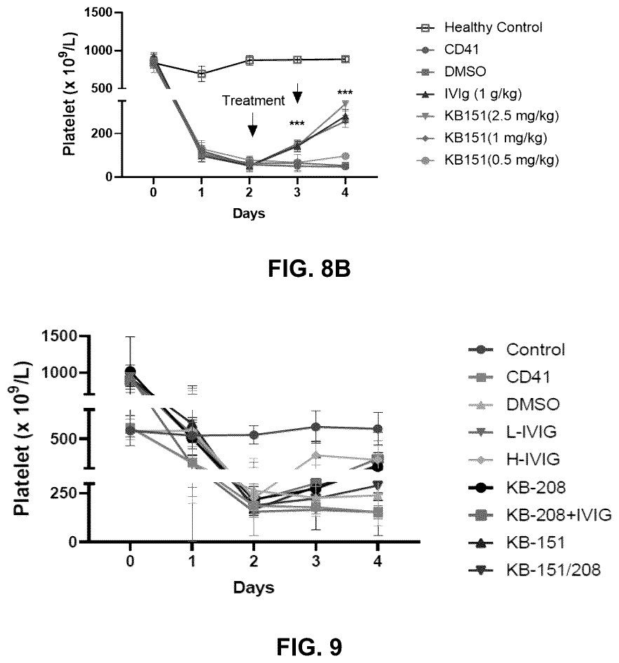

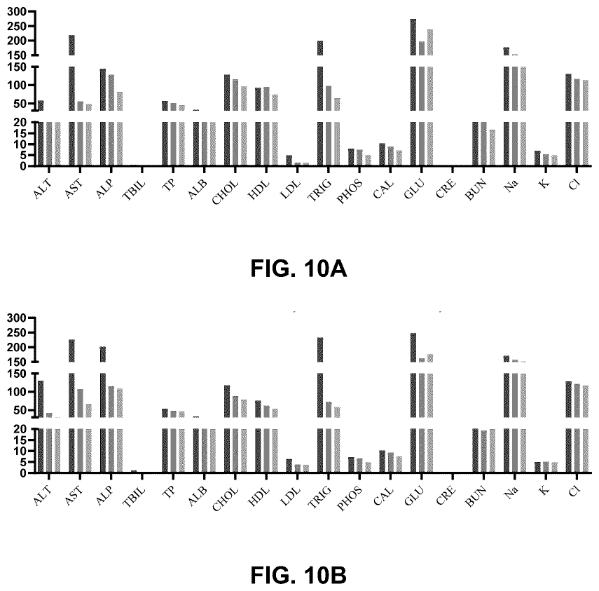

The efficacy of KB-151 and KB-208 at diminishing the platelet level was assessed in three ITP mice models: C57BL/6 (FIG. 7), Balb/c (FIGS. 8A-8B), and CD1 (FIG. 9). Similar efficacy at ameliorating ITP was observed in the three models. The toxicity was also assessed using the Balb/c model. Blood obtained from the mice after 60 days of treatment with KB-151 was analyzed and the biochemistry is shown in FIGS. 10A-10B. Minimal toxicity was observed. Moreover, a histopathology performed on the liver, heart, spleen, lymph nodes, and kidney of the mice showed abnormalities nor any toxicity.

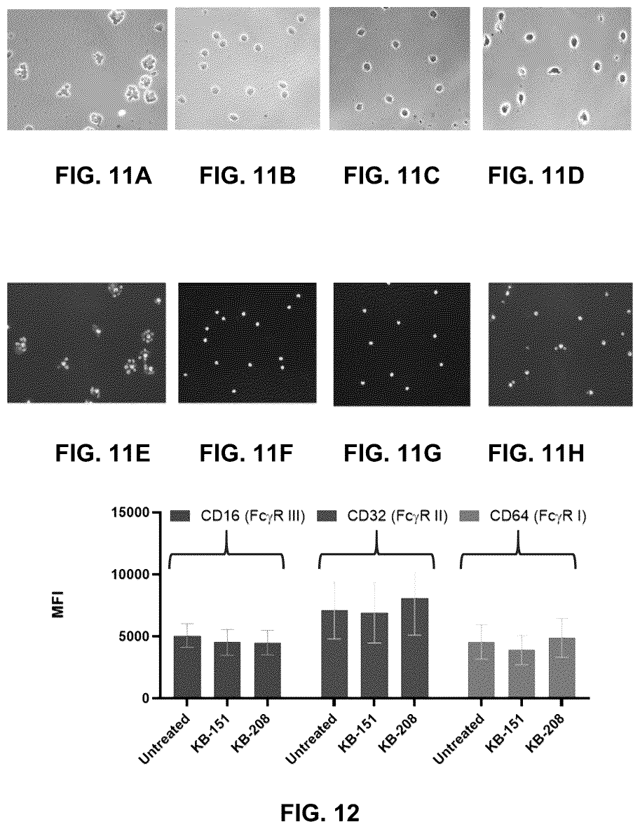

Fluorescent dye-labelled RBCs opsonized with anti-D were used to study the mechanism of phagocytosis inhibition using a room temperature assay to look for RBC rosettes (attachment of antibody-opsonized RBCs' to FcγRs) in the presence of monocytes. IVIg was used as the positive control and no treatment as the negative control. In the negative control, monocytes bound to the RBC and initiated phagocytosis (FIGS. 11A and 11E). However, KB-151 and KB-208 were found to inhibit the binding and inhibit phagocytosis similarly to IVIg (FIGS. 11B-11D and 11F-11H).

Due to observing similar results as IVIG, the binding of KB-208 and KB-151 to FcγR was evaluated to determine the attachment between KB-208 or KB-151 and FcγR. Three monoclonal antibodies each specific for FcγRI (CD64), FcγRII (CD32), and FcγRIII (CD16) were used to test the resulting expression of FcγRI, FcγRII and FcγRIII. The test was performed on monocyte cells and the results are presented in FIG. 12. As can be observed from FIG. 12, the mechanism of action of KB-208 and KB-151 to attach to opsonized red blood cells is independent of binding the three tested Fcγ receptors.

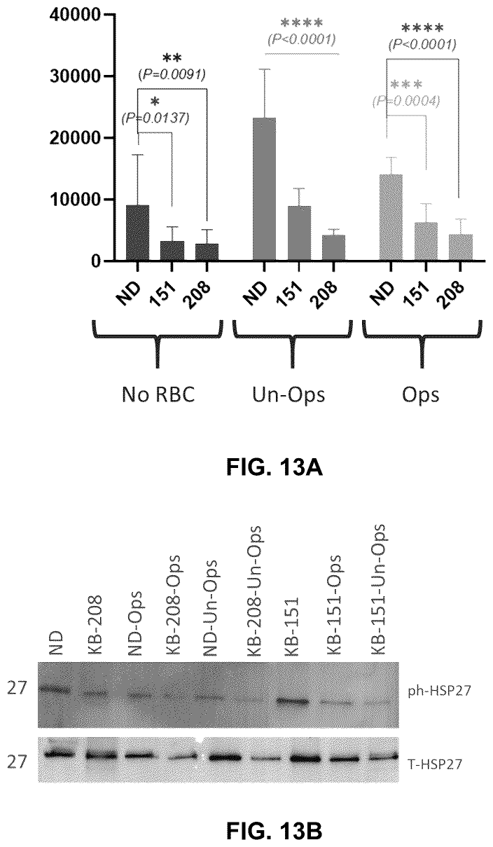

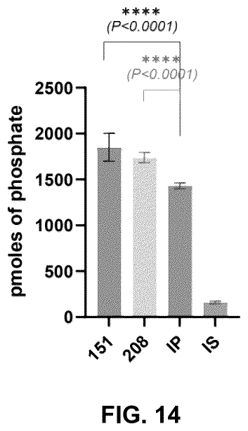

The mechanism of action of KB-208 and KB-151 was investigated further to evaluate the effects on signal transduction. Many kinases were studied including Akt1/2/3 (T308), Akt1/2/3 (S473), CREB, EGF R₁ eNOS, ERK1/2, Chk-2, c-Jun, Fgr, GSK-3a/p, GSK-3p, HSP27, p53 (S15), p53 (S46), p53 (S392), JNK 1/2/3, Lck, Lyn, MSK1/2, p70 S6 kinase (T389), p70 S6 kinase (T421/S424), PRAS40, p38a, PDGF RP, PLC-γ1, Src, PYK2, RSK1/2, RSK1/2/3, STAT2, STAT5a/b, WNK1, Yes, STAT1, STAT3 (Y705), STAT3 (S727), β-catenin, STAT6, and HSP60. The most significant effect was found on HSP27 and it was found that the KB-208 and KB-151 dephosphorylate HSP27 (FIGS. 13A-13B). It was then confirmed that KB-208 and KB-151 activate PP2A (in other words they increase its phosphatase activity) (FIG. 14). PP2A is a serine phosphatase that dephosphorylates HSP27. Deregulation of HSP27 also occurs in diseases other than immune cytopenias for example prostate cancer, colorectal cancer, and breast cancer.

In conclusion, after screening more than 200 different chemical compounds, the present work identified four pyrazole core compounds (KB-151, KB-208, KB-198, and KB-210), that demonstrated the ability to inhibit the phagocytosis due to an immune mediated mechanism.

1. A compound of formula I 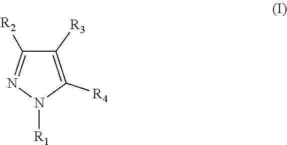
wherein R₁ is phenyl or ethyl;
R₂ is selected from the group consisting of H, —C(O)—O—CH₂—CH₃ and 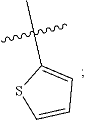 [SMILES: *c1cccs1]
R₃ is H, 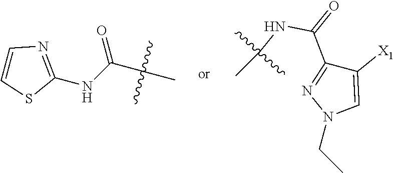
wherein X₁ is H, methyl or a halogen such as Br, F or Cl; and
R₄ is —CN or 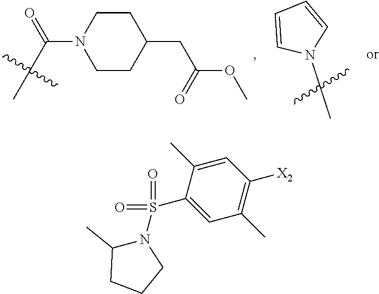
wherein X₂ is H or methyl;
wherein when R₁ is phenyl
R₂ is —C(O)—O—CH₂—CH₃, R₃ is 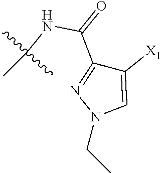
wherein X₁ is H, methyl or a halogen such as Br, F or Cl, and R₄ is —CN;
R₂ is 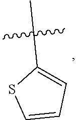 [SMILES: *c1cccs1]
R₃ is H and R₄ is 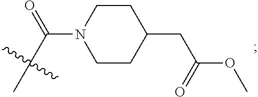 [SMILES: *C(=O)N1CCC(CC(=O)OC)CC1]
or
R₂ is H, R₃ is 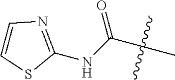 [SMILES: *C(=O)Nc1nccs1]
and R₄ is 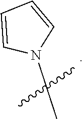 [SMILES: *n1cccc1]
and when R₁ is ethyl R₂ and R₃ are H and R₄ is 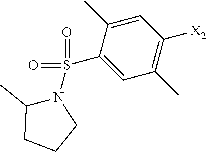
wherein X₂ is H or methyl.

2. The compound of claim 1, wherein the compound of formula I is

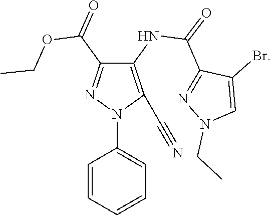 [SMILES: CCOC(=O)c1nn(-c2ccccc2)c(C#N)c1NC(=O)c1nn(CC)cc1Br]

3. The compound of claim 1, wherein the compound of formula I is

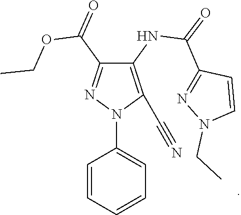 [SMILES: CCOC(=O)c1nn(-c2ccccc2)c(C#N)c1NC(=O)c1ccn(CC)n1]

4. The compound of claim 1, wherein the compound of formula I is

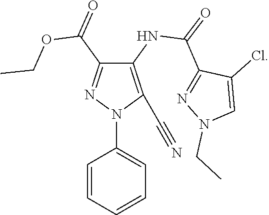 [SMILES: CCOC(=O)c1nn(-c2ccccc2)c(C#N)c1NC(=O)c1nn(CC)cc1Cl]

5. The compound of claim 1, wherein the compound of formula I is

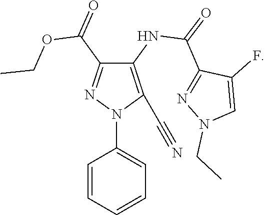 [SMILES: CCOC(=O)c1nn(-c2ccccc2)c(C#N)c1NC(=O)c1nn(CC)cc1F]

6. The compound of claim 1, wherein the compound of formula I is

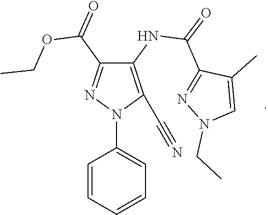 [SMILES: CCOC(=O)c1nn(-c2ccccc2)c(C#N)c1NC(=O)c1nn(CC)cc1C ; "."]

7. The compound of claim 1, wherein the compound of formula I is

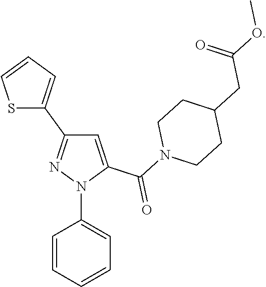 [SMILES: COC(=O)CC1CCN(C(=O)c2cc(-c3cccs3)nn2-c2ccccc2)CC1]

8. The compound of claim 1, wherein the compound of formula I is

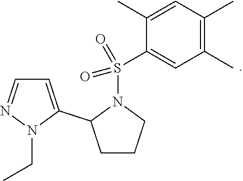 [SMILES: CCn1nccc1C1CCCN1S(=O)(=O)c1cc(C)c(C)cc1C]

9. The compound of claim 1, wherein the compound of formula I is

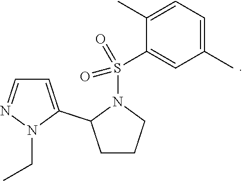 [SMILES: CCn1nccc1C1CCCN1S(=O)(=O)c1cc(C)ccc1C]

10. The compound of claim 1, wherein the compound of formula I is

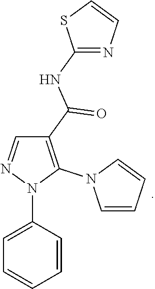 [SMILES: O=C(Nc1nccs1)c1cnn(-c2ccccc2)c1-n1cccc1]

11. A method of treating an immune cytopenia in a subject in need thereof, the method comprising administering a therapeutically effective amount of the compound of formula I as defined in claim 1.

12. A method of treating an immune cytopenia in a subject in need thereof, the method comprising administering a therapeutically effective amount of a pharmaceutical composition comprising the compound of formula I as defined in claim 1 and a pharmaceutically acceptable excipient.

13. The method of claim 11, wherein the immune cytopenia is immune thrombocytopenia, hemolytic disease of the fetus and newborn, autoimmune hemolytic anemia, alloimmune hemolytic anemias, delayed hemolytic transfusion reaction or autoimmune neutropenia.

14.-20. (canceled)

21. A method of dephosphorylating HSP27 in a subject in need thereof, the method comprising administering to the subject in need thereof a therapeutically effective amount of the compound of claim 2.

22. A method of dephosphorylating HSP27 in a subject in need thereof, the method comprising administering to the subject in need thereof a therapeutically effective amount of composition comprising the compound as defined in claim 2 and a pharmaceutically acceptable excipient.

23. The method of claim 21, wherein the subject has a cardiovascular disease or a cancer where HSP-27 is deregulated and over phosphorylated.

24. The method of claim 23, wherein the cardiovascular disease is atherosclerosis.

25. The method of claim 23, wherein the cancer is prostate cancer, colorectal cancer or breast cancer.

26.-30. (canceled)
